SEQUENCE LISTING (13 sequences)

SEQ ID NO: 1 - DNA, 47 bp, Artificial sequence
  Partly modified native attP sequence
gtgccccaactggggtaacctttgagttctctcagttgggggcgtag

SEQ ID NO: 2 - DNA, 70 bp, Artificial sequence
  Partly modified native attB sequence
ctcgaagccgcggtgcgggtgccagggcgtgcccttgggctccccgggcgcgtactccacctcacccatc

SEQ ID NO: 3 - DNA, 47 bp, Artificial sequence
  Partly modified native attP sequence
gtgccccaactggggtaacctaagagttctctcagttgggggcgtag

SEQ ID NO: 4 - DNA, 70 bp, Artificial sequence
  Partly modified native attB sequence
ctcgaagccgcggtgcgggtgccagggcgtgcccaagggctccccgggcgcgtactccacctcacccatc

SEQ ID NO: 5 - DNA, 1941 bp, Artificial sequence
  Partly modified native PhiC31 gene
atgaccatgattaccccatctgcccagctgaccctgacaaagggcaataagagctggtctagcctggtgacagctgcttctgtgctggagtttgccaccatgatccaaggggtcgctggggaagtgacttatgccggggcgtacgaccgtcagtctcgggagcgcgagaactctagcgcggcgtctccggccactcagcgtagcgctaacgaggccaaagccgccgctctccagcgcgagatcgagcgcgccgggggccggtttcgtttcgtcggtcacttcagcgaggcccccggcacatctgccttcggtacagccgagcgccctgagttcgaacgcattctgaacgaatgccgcgccggtcggctgaacatgattatcgtgtatgacgtgtctcgcttctctcgcctgaaggttatggacgccatccctatcgtgtcagaattactggccctgggcgtgacaatcgtctctacgcaggaaggcgtgttcagacaagggaacgttatggacctgatccacctgatcatgcggctggacgcctctcacaaagaaagctctctgaagtctgccaagatcctggacacaaagaacctccagcgcgaacttggcggttacgtgggcgggaaggccccctacggcttcgagcttgtcagcgagacaaaggagattacacgcaacggacgtatggtcaatgtggttatcaacaagctcgcccactctaccacgcctctcaccggacctttcgagttcgagccagacgtaattcggtggtggtggcgtgagatcaagacacacaaacacctccctttcaagcctggcagtcaagccgccatccaccctggctctattaccggactctgtaagcgcatggacgcggacgccgtgcctaccagaggcgagacaatcgggaagaagaccgcgtcgtctgcctgggaccctgcgaccgtcatgcgtattctcagagaccctcgtatcgccgggttcgctgcggaggtgatttacaagaagaagccagacggcacacctaccacaaagatcgagggataccgcatccagcgcgaccctattactctgcggcctgtggagcttgattgcggtcctattatcgagcctgcggagtggtatgagcttcaggcctggttggacggacgtggtcgcggcaagggtctctctcggggtcaagccatcctgtctgctatggacaagctgtactgcgagtgtggcgccgttatgacgagcaagcgcggggaagaatctatcaaggacagttaccgctgccgtcgcagaaaggtggtggacccttctgcgcccggtcagcacgaaggcacttgcaacgtctctatggccgcgctggacaagttcgtcgccgaacgcattttcaacaagatccgtcacgccgaaggcgacgaagagacacttgccctcctgtgggaagccgcccgtcgcttcggcaagctcacggaggcccccgagaagtctggcgaaagagccaacctcgtcgccgagcgcgccgacgccctgaacgccctcgaagagctgtacgaagaccgcgctgcgggcgcctacgacggtcctgtcggacgaaagcacttcagaaagcaacaggcggccctgactctgcgccagcaaggtgccgaagagagactcgccgaactcgaagccgccgaagccccaaagctccctctcgaccaatggttcccagaagacgccgacgcggaccctaccggccccaagtcttggtggggtcgcgcctcggtagacgacaagcgcgtgttcgtgggtctgttcgtagacaagattgtcgttacaaagtctacgacaggccgtgggcaggggacacctatcgagaagcgcgcgtctattacttgggccaagcctcctaccgacgacgacgaagacgacgcccaggacggcacagaagacgtagctgcttgataa

SEQ ID NO: 6 - DNA, 34 bp, Artificial sequence
  Partly modified native lox gene
ataacttcgtataggatactttatacgaagttat

SEQ ID NO: 7 - DNA, 1031 bp, Artificial sequence
  Partly modified native Cre gene
atgtcaaaccttctcaccgtccaccaaaacctccccgcactccccgttgacgccacctccgacgaggtcagaaaaaacctcatggacatgttccgggaccgccaggccttttccgaacacacttggaaaatgcttctcagcgtttgccgtagttgggccgcttggtgtaaactcaacaaccgcaagtggttccccgccgaacccgaggacgtccgcgattaccttctgtatttgcaagcgcgaggactggccgtgaaaaccatccagcaacatctgggtcagcttaacatgttgcaccggaggagcggcctgccacggcctagcgactccaacgcggtgtccctcgtgatgaggagaatccgcaaggagaatgtggacgccggagaaagagcaaagcaggccctggccttcgagaggactgacttcgaccaagtccggtcgctgatggagaactcggaccgatgtcaggacatcaggaacctcgcattctcggcattgcctacaacaccctgctgagaattgcagagatcgcccgcatccgcgtcaaggacatttcgagaaccgacggagggcggatgctgattcacatcggcaggactaagaccctcgtgtcaaccgccggagtggaaaaggccctcagcctgggagtgacaaagctcgtggagcgctggatctccgtgtcgggggtggccgacgatccgaacaattacctgttctgccgggtccgcaaaaatggggtggccgccccgtctgctacaagccagttgtccactcgcgccctggaaggaatcttcgaggccacgcaccgcctgatctatggggcaaaggacgattccggccagaggtatctcgcgtggtccggtcactccgcgcgcgtgggcgcggcccgggacatggcccgggctggagtgtccatccctgaaatcatgcaggccggtggatggaccaacgtgaacatcgtgatgaactacattcggaacctggacagcgaaactggtgctatggtccgcctgctggaggacggagattga

SEQ ID NO: 8 - DNA, 1428 bp, Artificial sequence
  Enhanced Green Fluorescent Protein fused to an FC from IgG1 (FC-eGFP gene)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccacgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaggttcctccagttccggcagctccagttccggtatgagtaaaggagaggaactcttcaccggagtcgtcccgatactcgtcgagctagacggagacgtcaacggccacaaattctccgtctccggcgagggggagggggacgccacctacggaaaactcacccttaagtttatttgcactaccggaaaactccccgtcccttggccaaccctagtcaccacgctgacatacggagtccaatgtttctcgcggtatcccgaccacatgaagcagcatgactttttcaaatccgcgatgcctgagggctacgtgcaggaacgcaccatcttcttcaaggacgacgggaattacaagactagagccgaggtcaagtttgaaggagacaccctcgtgaatcgcatcgagcttaagggcattgacttcaaggaggacggcaacatcctgggtcacaagctggagtacaactacaactcgcataacgtctacatcatggccgacaagcaaaagaacggtatcaaggtcaacttcaagattaggcacaacattgaggatgggtccgtccaactggccgaccactaccagcagaacacccccatcggcgacggacctgtgctcctgcctgataaccactatctcagcactcagagcgcactgtccaaggaccctaacgaaaaacgggaccacatggtcttgctggagttcgtgacagccgctggtattaccctgggcatggatgaactgtataag

SEQ ID NO: 9 - DNA, 1431 bp, Artificial sequence
  Blue Fluorescent Protein variant fused to an FC from IgG1 (FC-TagBFP2 gene)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccacgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaggttcctccagttccggcagctccagttccggtatggtgtcgaagggagaggagctgattaaggagaacatgcacatgaagctgtatatggaagggacggtggacaaccaccacttcaagtgcaccagcgaaggagaaggaaagccttacgaaggcactcaaactatgcggatcaaagtggtggaaggcggtcctcttccgttcgccttcgacatcttggccacctccttcctctacggctccaagacctttatcaaccacacccagggaatcccggacttctttaagcagagcttccctgagggcttcacctgggaaagagtgacaacctacgaggacggtggcgtcctgaccgcgacccaggacacctccctgcaagacggctgcctgatctacaacgtcaagattcgcggcgtgaacttcacctccaatggtccagtgatgcagaagaaaactctgggatgggaggccttcactgaaactctgtaccccgccgatggaggactggaggggaggaacgatatggctttgaagctcgtggggggatcgcacctgattgcgaatgccaagaccacctacagatccaagaaacccgccaagaacctcaagatgcccggagtctactacgtggactatagactggaacggatcaaggaagccaacaacgagacttacgtggaacagcacgaggtcgctgtggcacgctactgtgatctgccgtcaaagctcgggcataagctcaactgataa

SEQ ID NO: 10 - DNA, 738 bp, Artificial sequence
  Red Fluorescent Protein Variant (TagRFP-T gene)
atggtgtcaaagggagaggaactgattaaggagaatatgcacatgaaactctacatggaggggaccgtgaacaaccaccacttcaagtgcacctccgagggcgaagggaagccgtacgagggaactcagaccatgcggattaaggtcgtcgaagggggtcctctgccattcgccttcgacatcctcgccacatcctttatgtacggatcgcggaccttcatcaaccacactcagggtatccccgacttcttcaagcaatcgttcccggaaggctttacttgggagcgcgtgaccacctacgaggatggaggggtgctgacggccactcaggacaccagcctgcaagacggctgtcttatctacaacgtgaagattcgcggcgtgaacttccctagcaacggtccggtcatgcagaaaaagaccctgggttgggaggctaacaccgaaatgctctatcctgcggacggaggattggaaggccggactgacatggccctgaaacttgtgggcggcggacatctgatctgcaatttcaagaccacttaccgctccaagaagcccgccaagaacctgaagatgcctggagtgtactacgtggaccacagactcgaaaggatcaaggaggcggataaggaaacctacgtggaacagcatgaagtggcagtggccagatactgcgatctgccgtccaagctcggccacaagctgaacggaatggacgagctgtataagtgataa

SEQ ID NO: 11 - DNA, 714 bp, Artificial sequence
  Enhanced Green Fluorescent Protein (eGFP gene)
atgagtaaaggagaggaactcttcaccggagtcgtcccgatactcgtcgagctagacggagacgtcaacggccacaaattctccgtctccggcgagggggagggggacgccacctacggaaaactcacccttaagtttatttgcactaccggaaaactccccgtcccttggccaaccctagtcaccacgctgacatacggagtccaatgtttctcgcggtatcccgaccacatgaagcagcatgactttttcaaatccgcgatgcctgagggctacgtgcaggaacgcaccatcttcttcaaggacgacgggaattacaagactagagccgaggtcaagtttgaaggagacaccctcgtgaatcgcatcgagcttaagggcattgacttcaaggaggacggcaacatcctgggtcacaagctggagtacaactacaactcgcataacgtctacatcatggccgacaagcaaaagaacggtatcaaggtcaacttcaagattaggcacaacattgaggatgggtccgtccaactggccgaccactaccagcagaacacccccatcggcgacggacctgtgctcctgcctgataaccactatctcagcactcagagcgcactgtccaaggaccctaacgaaaaacgggaccacatggtcttgctggagttcgtgacagccgctggtattaccctgggcatggatgaactgtataag

SEQ ID NO: 12 - DNA, 717 bp, Artificial sequence
  Blue Fluorescent Protein variant (TagBFP2 gene)
atggtgtcgaagggagaggagctgattaaggagaacatgcacatgaagctgtatatggaagggacggtggacaaccaccacttcaagtgcaccagcgaaggagaaggaaagccttacgaaggcactcaaactatgcggatcaaagtggtggaaggcggtcctcttccgttcgccttcgacatcttggccacctccttcctctacggctccaagacctttatcaaccacacccagggaatcccggacttctttaagcagagcttccctgagggcttcacctgggaaagagtgacaacctacgaggacggtggcgtcctgaccgcgacccaggacacctccctgcaagacggctgcctgatctacaacgtcaagattcgcggcgtgaacttcacctccaatggtccagtgatgcagaagaaaactctgggatgggaggccttcactgaaactctgtaccccgccgatggaggactggaggggaggaacgatatggctttgaagctcgtggggggatcgcacctgattgcgaatgccaagaccacctacagatccaagaaacccgccaagaacctcaagatgcccggagtctactacgtggactatagactggaacggatcaaggaagccaacaacgagacttacgtggaacagcacgaggtcgctgtggcacgctactgtgatctgccgtcaaagctcgggcataagctcaactgataa

SEQ ID NO: 13 - DNA, 792 bp, Artificial sequence
  Neomycin resistance gene (NeoR gene)
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttc

(19) United States
(12) Patent Application Publication
(10) Pub. No.: US 2023/0159958 A1
(43) Pub. Date: May 25, 2023

(54) METHODS FOR TARGETED INTEGRATION

(71) Applicant: CYTIVA SWEDEN AB, Uppsala (SE)

(72) Inventors: Andreas Jonsson, Uppsala (SE); Daniel Ivansson, Uppsala (SE)

(21) Appl. No.: 17/995,571

(22) PCT Filed: Apr. 6, 2021
(86) PCT No.: PCT/EP2021/058952

§ 371 Date: Oct. 5, 2022

(30) Foreign Application Priority Data
Apr. 8, 2020 (GB) 2005180.1

(51) Int. Cl. C12N 15/90; C12N 9/12; C12N 15/85

(52) U.S. Cl. CPC C12N 15/907; C12N 9/12; C12N 15/85; C12N 2800/30; C12N 2800/106

(57) ABSTRACT

The present disclosure relates to a method for targeted integration of a donor vector into a specific pre-defined genomic location of an isolated eukaryotic host cell. The vector and host cell together comprise nucleic acid components allowing for the selection of cells having integrated the donor vector into the pre-defined genomic location of the host cell. In addition, it provides for the identification of any random integrations of the donor vector(s) into other parts of the host cell genome. Once identified, such cells present an excellent alternative for subsequent recombinant protein production.

### TECHNICAL FIELD OF THE INVENTION

The present invention relates to the field of optimized expression systems for the production of recombinant proteins. More specifically, it relates to a cell-based method utilizing targeted integration of a donor vector into a specific pre-defined genomic location of a eukaryotic host cell genome, wherein said vector and host cell comprises nucleic acid components rendering it possible to selectively choose those cells having integrated the donor vector into a pre-defined genomic location of the host cell genome and to detect and remove cells having undergone any additional random integration events into other parts of the genome.

### BACKGROUND OF THE INVENTION

During the last 30 years recombinant protein therapeutics has evolved from a novelty to a dominating position among marketed drugs. Recombinant production of therapeutic proteins has surpassed the 100 billion $ per year market volume and plays an important role in the global economy as well as in advanced medical care. The therapeutic protein class includes replacement proteins (insulin, growth factors, cytokines and blood factors), vaccines (antigens, VLPs) and monoclonal antibodies. The by far dominating format is the monoclonal antibodies. Some of the recombinant proteins can be produced in simple microbial cells such as E. coli, but for more complex proteins including the monoclonal antibody class Chinese Hamster Ovary (CHO) cells is the dominating host for production [1, 2].

The dominating approach to generate a high performance therapeutic protein producing cell line within the industry today is to introduce the recombinant protein genes into the genome of a host CHO cell line via a random integration approach and select/screen for individual cells having integrated the genes at active genomic sites at a copy number yielding sufficiently high transcription and that at the same time having a phenotype capable of supporting high protein translation and secretion. This is a highly work intensive and time-consuming process with large inherent uncertainties and biological variation. Typical process duration spans between 3-12 months depending on the growth of the host cells, the level of automation implemented and the end point (for example if assessment of long-term clone stability is included).

One fundamental limitation associated with the random integration approach is the low sampling of the cellular diversity in a transfected pool of cells. Only around 0.1-1% of the transfected cells integrate recombinant DNA. Further, this sub-population is highly heterogeneous in terms of integration locations, copy number and integrity of the integrated DNA. Adding the inherent global phenotypic variation of CHO cells, which is inherent for CHO cells due to the high genomic and epi-genomic plasticity, makes finding a high producing clone like finding a needle in a haystack. This also explains why a high variation in protein production from non-clonal stable pools is generally observed (stochastic sampling of phenotypic diversity).

This under-sampling and high biological noise also make comparison of different gene cassette designs of a therapeutic protein candidate for optimization of expression difficult. Comparison of multiple variants via parallel generation of stable pools is highly work intensive and the high biological noise will make results unreliable. Use of simultaneous transfection of variant libraries is hampered by the fact that random integration typically results in integration of multiple copies of an expression vector and hence any cell generated through such a workflow will typically contain integrated copies from more than one gene cassette design. Improving protein expression by Cell Line engineering strategies based on random integration of effector genes is hampered by the same reasons.

One potentially major improvement to all of the above limitations is to utilize targeted integration (Site-Directed Integration; SDI) of Genes of Interest (GOI's). In such a scenario a pre-identified genomic location known to support high and stable transcription is used as a target destination for GOI's. Using intelligent combinations of pre-introduced sequences and vector designs, including the use of co-transfected nucleic acid enzymes such as nucleases or recombinases, will facilitate targeted insertion and ensure that all cells in culture will contain correctly inserted GOIs and hence have a high transcription rate. This will significantly reduce the number of clones needed in a screening campaign for Cell Line Development (CLD) and reduce biological noise in comparisons of gene cassette designs or Cell Line engineering efforts. Multiple technical solutions for targeted integration are described in the art [3-6]. However, despite so, challenges remain.

A general challenge for all strategies utilizing targeted integration is to generate high enough expression of the GOI as typical solutions result in the integration of a single GOI copy. Other challenges and limitations of available solutions described in the art are outlined below.

The Flp-In system (based on the Flp/Flippase recombinase, also referred to as Flippase recombinase) for targeted integration [7] is an example of a solution utilizing a single recombinase recognition sequence in combination with it's recombinase to enable targeted integration at a pre-defined genomic location. Following the action of the recombinase the complete expression vector is integrated at the recombinase recognition sequence. Cells with correct integration events can be selected as integration at the recombinase recognition site inactivates one selection marker and activates a second selection marker. Major drawbacks with this solution are (i) there is no mechanism to detect or remove cells having integrated additional copies of the expression vector by random integration events, (ii) there is no mechanism to remove sequence regions, such as plasmid backbone sequences and active selection marker genes, that can be negative to the expression of the GOI and (iii) the method has questionable flexibility in the choice of selection marker as activation of the selection marker during integration results in the fusion of extra amino acids at the N-terminal that can impact it's functionality.

To avoid presence of sequences with a potential negative impact on GOI expression following targeted integration, different solutions for cassette exchange reactions at a pre-defined genomic location has been described in the art [3-6]. An example of such a solution has been disclosed by Rentschler [8]. The pre-defined genomic location utilizes an active selection marker gene (GFP) flanked by two orthogonal recombinase recognition sequences both targets for the same recombinase. The GOI in the expression vector is in turn flanked by two recombinase recognition sequences matching the two present in the genome. Upon action of the recombinase, cassette exchange between the selection marker cassette and the GOI cassette can occur. Cells having undergone the cassette exchange can be selected by absence of GFP expression. Drawbacks for this kind of solution are (i) there is no mechanism to detect or remove cells having integrated additional copies of the expression vector by random integration events, (ii) as selection of cells having undergone cassette exchange is based on absence of an initially active gene product, the time point for selection must be delayed to allow for degradation/dilution of GFP.

Haghighat-Khah R E, et al. discloses a two-step site-specific cassette exchange system in insects, i.e. the Aedes aegypti Mosquito and the Plutella xylostella moth [9]. The exchange system utilizes a phiC31 recombinase for integration of an expression vector at a pre-defined genomic location followed by the use of a second recombinase (Cre or Flp) for excision of plasmid backbone sequences. However, the exchange system of Haghighat Khah R E, et al. does not provide means for distinguishing between targeted integration and random integration events. In addition, no means to remove the selection marker gene are provided.

Yuan, Y; et al. discloses a recombinase-based method to produce selection marker- and vector-backbone-free transgenic cells utilizing PhiC31-mediated gene delivery into pseudo-attP sequences present naturally in the genome of the targeted cells [10]. Selection of cells in which integration has occurred is achieved via presence of an active eGFP expression cassette in the expression vector and an att-B-TK fusion gene becoming inactivated upon targeted integration was used as a negative selection marker to eliminate random integration events in a second selection step. The selection system and the plasmid bacterial backbone was subsequently excised by using the two other recombinases Cre and Dre. Critical drawbacks in the method disclosed by Yuan, Y; et al. for adoption to recombinant protein production applications based on integration into one pre-defined genomic location are (i) the method does not provide means to distinguish between cells having undergone integration only at the pre-defined location from cells having undergone integration both at the pre-defined site and a random pseudo-attP site as inactive TK genes would result from both scenarios, (ii) the first selection step cannot be performed until transient expression of the selection marker has vanished which adds time, (iii) the first selection step does not distinguish between desired integration, integration at a pseudo-attP site or a random integration event.

Accordingly, there is still a need in the art to identify improved expression systems for the production of recombinant proteins. In this regard, there is a need in the art for improving existing SDI systems to combine the following desirable features into a single solution:
(a) Ability to support GOI expression levels in pair with random integration solutions,
(b) Fast and specific selection of cells having undergone integration at the pre-defined genomic location,
(c) Means to detect and remove cells having undergone additional non-desired integration events,
(d) Means to avoid presence of sequences that can have a negative effect on GOI expression in isolated cells.

### SUMMARY OF THE INVENTION

The above-mentioned problems have now been solved or at least mitigated by the provision of methods and means presented further herein.

The present disclosure provides a novel solution for recombinant protein production utilizing Site Directed Integration (SDI) of a single copy of a donor vector into a pre-defined genomic location of an isolated eukaryotic host cell. The SDI-based system of the present disclosure is based on a unique and inventive combination of well-established nucleic acid components for the efficient integration of a donor vector into a dedicated target site of the host cell. The method provides for the specific positive selection of host cells having integrated the donor vector into the dedicated pre-defined genomic location. The method also provides for, by negative selection, detecting and optionally removing any cells for which undesired integration events have occurred in other locations of the host cell genome. This two-step selection method is unique and will be very useful in the field of recombinant protein production.

As mentioned previously herein, a critical component for improved cell line development, flexible cell line engineering and enabling of advanced applications such as simultaneous probing of gene construct libraries, is increased control of recombinant gene integration into host cell lines and better control over their copy number. This is now provided by the present disclosure.

Initially, Chinese Hamster Ovary (CHO) cells were used to set up a method presented herein as putative hot spot locations has been identified, but the SDI system should be applicable to any eukaryotic cell system including mammalian cells, such as human cells.

Accordingly, in a first aspect, the present disclosure relates to a method for targeted integration of a donor vector into a pre-defined genomic location of a eukaryotic cell, said method comprising:

i) Providing a eukaryotic cell comprising a pre-defined genomic location, which pre-defined location comprises:

a. nucleic acid sequence I1 comprising a recognition site for a first DNA enzyme;

b. a nucleic acid sequence E1 comprising a recognition site for a second DNA enzyme; and

c. a promotor nucleic acid sequence P1;

ii) Providing a donor vector comprising:

a. a nucleic acid sequence I2;

b. a nucleic acid sequence of interest;

c. a nucleic acid sequence E2 comprising a recognition site for said second DNA enzyme;

d. a nucleic acid sequence encoding a first selection marker; and

e. optionally an expression cassette encoding a second selection marker;

iii) Contacting the donor vector with the cell in the presence of a first DNA enzyme, wherein the presence of the first DNA enzyme enables recombination between the nucleic acid sequence I2 of the donor vector and the nucleic acid sequence I1 present in the pre-defined genomic location of the cell;

iv) Selecting a cell having the donor vector integrated at the pre-defined genomic location by detecting the expression of the first selection marker in the cell, wherein the expression of the first selection marker is activated by the promotor nucleic acid sequence P1 at the pre-defined genomic location; and

v) Isolating the cell selected in the preceding step.

In a further aspect, the present disclosure relates to an isolated eukaryotic cell obtainable by a method as described herein.

In yet a further aspect, the present disclosure relates to the use of an isolated eukaryotic cell obtainable by a method as described herein for the production of a recombinant protein.

In yet a further aspect, the present disclosure relates to a method for producing a recombinant protein, said method comprising:

i) obtaining an isolated eukaryotic cell comprising a donor vector comprising one or more nucleic acid sequences of interest integrated at a pre-defined genomic location by performing the method as disclosed herein, wherein at least one nucleic acid sequence of interest comprises at least one expression cassette comprising a gene encoding a protein of interest;

ii) in said cell of step i), producing a protein encoded by the gene of interest; and

iii) isolating the protein of step ii).

### BRIEF DESCRIPTION OF THE DRAWINGS

FIG. 1 shows a schematic illustration of the general concept of a method for targeted integration of a donor vector into a pre-defined genomic location of a host cell via the use of at least two DNA enzymes with orthogonal specificity.

FIG. 2 shows a schematic illustration of the Landing Pad designs (nucleic acid sequences present in the pre-defined genomic locations of the host cell genome) and matching Donor Vectors for the HyClone LP1P1 and HyClone LP2P2 cell lines.

FIG. 3 illustrates an example of Flow Cytometry plots at day 7 post transfection in comparison to a non-transfected control (NC). Density of cells in plots having a concentrated main population is visualized by alternating black and white regions (20% of total cells in each region). Upper row shows FACS data for a non-transfection control (NC) culture of HyClone CHO cells. Middle row shows FACS data from a random integration control (RI) based on HyClone CHO cells (lacking LP) transfected with the Donor Vector B only (without PhiC31). Lower row shows FACS data for the HyClone CHO LP2P2 cell line transfected with PhiC31 and Donor Vector B (SDI). The gate (B, D, F) in middle plot of each row is set based on the non-transfected control and reports percentage of cells having activated the selection marker above background.

FIG. 4 shows a schematic illustration of the Landing Pad Cell line and Donor Vector used as well as the alterations at the Landing Pad expected to occur through the activity of PhiC31 (1) and Cre (2).

FIG. 5 shows flow cytometry plots of SDI populations at day 7 post transfection of Cre recombinase variants in comparison to a negative mock transfection control. Density of cells in plots having a concentrated main population is visualized by alternating black and white regions (20% of total cells in each region). Upper panel shows plots for a mock transfection lacking Cre recombinase encoding nucleic acid molecules, middle panel shows plots of a population transfected with a Cre recombinase expression plasmid and the lower panel shows plots of a population transfected with synthetic Cre recombinase mRNA.

FIG. 6 shows a schematic illustration of the Landing Pad Cell line and Donor Vectors used as well as the alterations at the Landing Pad expected to occur through the activity of PhiC31 recombinase (1) and Cre recombinase (2).

FIG. 7 shows flow cytometry plots from the steps performed according to FIG. 6. Density of cells in plots having a concentrated main population is visualized by alternating black and white regions (20% of total cells in each region). The left plot in the upper panel show the population following the second eGFP (Green Fluorescent Protein) positive sort. The middle plot in the upper panel show the population 7 days post Cre recombinase transfection. The right plot in the upper panel show the population after the eGFP negative sort performed according to gate E following Cre recombinase transfection. The lower panel show plots of the population seven days post transfection of Step 2 Sort cells with DNA Donor Vector B.

FIG. 8 shows a schematic illustration of the Landing Pad Cell line and Donor Vectors used. GSx=Glutamine Synthetase gene variant.

FIG. 9 shows flow cytometry plots from the generation of a cell population using SDI of the Donor vector. Density of cells in plots having a concentrated main population is visualized by alternating black and white regions (20% of total cells in each region). Upper panel shows a plot of a population having undergone G418 selection, RFP (Red Fluorescent Protein) positive FACS sorting and transfection with synthetic Cre recombinase mRNA. eGFP histograms are shown for both the RFP negative sub-population (corresponds to integration at the Landing Pad with Cre recombinase mediated excision of TagRFP-T) and the RFP positive sub-population (corresponds to failed Cre recombinase mediated excision of TagRFP-T that can be caused by off-target integration or truncated integration at the Landing Pad). The lower panel shows plots of the final SDI pool generated using a FACS sort of RFP negative/GFP positive cells from the upper panel.

FIG. 10 shows the first selection marker of said donor vector linked to a gene coding for said second DNA Enzyme via an IRES element. Both the first selection marker and the second DNA Enzyme is activated upon integration at the pre-defined genomic location.

FIG. 11 shows when the pre-defined genomic region also comprises an expression cassette for said first DNA Enzyme, located so that upon integration of the donor vector at the pre-defined genomic location it becomes flanked by recognition sites for said second DNA Enzyme and hence can be removed in the presence of said second DNA Enzyme.

FIG. 12 exemplify a variant for targeted integration where a gene editing enzyme is used to catalyze integration of the donor vector into the pre-defined genomic location of the host cell genome.

FIG. 13 shows a variant for targeted integration where recombinase mediated cassette exchange (RMCE) is used to catalyze integration at the pre-defined genomic location of the host cell genome.

FIG. 14 shows a variant for targeted integration where a single recombinase recognition site pair is used to catalyze integration of the donor vector at the pre-defined genomic location of the host cell.

FIG. 15 shows the use of a single recombinase recognition site pair to catalyze integration at the pre-defined genomic location and were the promotor P1 present at the pre-defined genomic location is functionally fused to the 5′-part of a split intron.

### DETAILED DESCRIPTION OF THE INVENTION

The present disclosure will now be described more closely in association with the accompanying drawings and some non-limiting examples.

### Definitions

Details of the present disclosure are set forth below. Although any materials and methods similar or equivalent to those described herein can be used in the practice or testing of the present invention, the preferred materials and methods are now described. All words and terms used herein shall be considered to have the same meaning usually given to them by the person skilled in the art, unless another meaning is apparent from the context.

Compositions “comprising” one or more recited elements may also include other elements not specifically recited.

The singular “a” and “an” shall be construed as including also the plural.

“Expression” is used to mean the production of a protein from a gene and refers herein to and comprises the steps of “the central dogma” i.e. the successive action of transcription, translation and protein folding to reach the active state of the protein.

An “expression vector” as defined herein, is a vector comprising nucleic acid sequences to achieve protein expression from the vector when present in a host cell. The expression vector herein is used e.g. to introduce a specific gene of interest into a cell, to thereafter direct the cell machinery for protein synthesis to produce the protein of interest encoded by the gene of interest. An expression vector can contain an “expression cassette”, said expression cassette containing the nucleic acid sequences to facilitate protein expression. In addition, the vector may contain other nucleic acid sequence elements or components.

A “donor vector” as referred to herein, is a vector, preferably a DNA vector, comprising nucleic acid elements or components for facilitating integration of the vector into the pre-defined genomic location of the isolated eukaryotic host cell. The donor vector carries a nucleic acid sequence facilitating a recombination event with a nucleic acid sequence present in the pre-defined genomic location of the host cell, a nucleic acid sequence of interest optionally encoding a protein of interest, a recognition site for the second DNA enzyme and a nucleic acid sequence encoding a first selection marker. Optionally, it may also contain an expression cassette for a second selection marker. A “donor vector” may sometimes herein also simply be referred to as a “vector”. A “donor vector” may sometimes be in the form of an expression vector such as when the donor vector comprises an expression cassette encoding a second selection marker. More specifically, a donor vector described herein contains at least a nucleic acid sequence I2 for recombination with I1 present in the pre-defined genomic location of the eukaryotic cell. In addition, it comprises a nucleic acid sequence of interest, herein also referred to as a gene of interest (“GOI”) if said nucleic acid of interest encodes a protein of interest. It also comprises a nucleic acid sequence E2 comprising a recognition site for the second DNA enzyme which makes it possible to excise parts of the vector backbone once a stable integration of the donor vector has occurred in the pre-defined genomic location of the host cell. It also contains a nucleic acid sequence encoding a first selection marker (SM1), the expression which will only be activated if the donor vector has been integrated into the correct position in the pre-defined genomic location of the host cell. Finally, the donor vector optionally comprises an expression cassette encoding a second selection marker (SM2). Following action of the second DNA enzyme the second selection marker will only be expressed and possible to detect in a cell if a random integration event of the vector has occurred and is used in the second round of selection of the present method. A donor vector is preferably a DNA donor vector but is not limited thereto. A DNA donor vector is sometimes abbreviated “DDV”.

An “expression cassette” is a nucleic acid component forming part of an expression vector which contains all the elements needed for initiation of transcription and translation of the protein of interest. The gene of interest encoding the protein of interest also forms part of the expression cassette. The expression cassette contains e.g. a promoter, essential for the initiation of transcription, and other sequences facilitation transcription, such as enhancer sequences. Sometimes the term “integration cassette” is used herein which corresponds to the nucleic acid sequences from the donor vector that remains at the pre-defined genomic location after the action of the second DNA enzyme. An “integration cassette” may comprise an “expression cassette”.

Herein “a” gene of interest refers to the nucleic acid components needed to produce a protein of interest and as a protein of interest can comprise multiple polypeptide chains can also refer to multiple genes of interest that are present in the same expression cassette. An expression cassette containing multiple genes of interest can either utilize individual promotors to achieve transcription of individual genes or two or more genes can be transcribed as a common mRNA with individual genes separated by i.e. IRES elements. This is in line with that herein, whenever “a” is used, this may also refer to the plural. An example of when an expression cassette comprises more than one gene of interest is when an antibody is to be expressed from the gene of interest, e.g. wherein a light and a heavy chain antibody component are present as separate genes in the expression cassette.

An “intron” is a nucleic acid sequence of a gene that is removed by RNA splicing once transcribed and during production of the final RNA product. Introns are non-coding regions of an RNA transcript, or the DNA encoding it, which are eliminated by splicing before translation.

Herein, a promotor functionally fused to the 5′-part of a split intron means that the transcription of the 5′-part of the split intron is driven by said promotor. Herein, the 5′-part of a split intron is defined as comprising a splice donor site sequence (such as GT). Herein, the 3′-part of a split intron may be defined as comprising (i) a splice branch site sequence, (ii) a Py-rich sequence region and (iii) a splice acceptor site sequence (such as AG).

Transcription comprises the conversion of DNA to RNA by the cell machinery. A “transcription regulatory sequence” is a segment of a nucleic acid sequence which is capable of increasing or decreasing the final expression of specific genes, i.e. by said sequences being capable of regulating the transcription of said gene. Examples of transcription regulatory sequences are promoters, enhancer and similar elements.

An untranslated region (“UTR”) refers to either of two sections on each side of a coding sequence on a strand of mRNA. On the 5′ side, it is called the 5′ UTR, on the 3′ side, it is called the 3′ UTR.

An upstream open reading frame (uORF), as referred to herein, is an open reading frame (ORF) within the 5′ untranslated region (5′UTR) of an mRNA molecule. uORFs are generally involved in the regulation of eukaryotic gene expression. Translation of the uORF typically inhibits downstream expression of the primary ORF (open reading fram), accordingly when present these cause reductions in protein expression. About half of the human genes contain these regions.

An Internal Ribosome Entry Site (“IRES”) is an RNA element that allows for translation initiation in a cap-independent manner. They are often referred to as distinct regions of RNA molecules that are able to recruit the eukaryotic ribosome to the mRNA. The location for IRES elements is often in the 5′ UTR region but it can also occur elsewhere in the mRNA.

A “plasmid” is a small circular extra-chromosomal DNA molecule that can replicate independently of the cell and are found in bacteria. Plasmids are often used as vectors for molecular cloning i.e. to transfer and introduce selected DNA to a host cell. Plasmids are built-up from specific and necessary elements and may contain genes that can be homo- or heterologous to the bacterial host cell. Plasmids contain e.g. always an bacterial origin of replication and most often a gene for specific antibiotics resistance.

A “nucleic acid sequence of interest” as referred to herein, may be defined as a nucleic acid sequence that one wishes to integrate into a cell to impact the functionality of said cell. It may comprise a gene of interest (“GOI”) that encodes a protein of interest.

By a “recombinant” protein as mentioned herein, is meant a protein manufactured from an expression cassette introduced into a cell by an expression vector. Techniques for producing recombinant proteins are well-known to the person skilled in the art.

A “promoter” is a region of DNA which initiates transcription of a gene upon the binding of RNA polymerase thereto. Promoters are located near the transcription start sites of a gene.

A “host cell” as referred to herein, relates to a eukaryotic cell which is intended to be or has been transformed by a donor vector as disclosed herein.

An “isolated cell”, “isolated host cell” or “isolated eukaryotic host cell” refers to a cell that has been isolated from its natural environment meaning that it is free from any additional components that may occur in nature and that it is not any longer part of its natural environment.

Herein, a “pre-defined genomic location” also sometimes referred to as a “Landing pad” (abbreviated as “LP”), or rather as a pre-defined genomic location comprising a Landing pad sequence, is intended to refer to a location, or a nucleic acid position, characterized by a particular nucleic acid sequence, in a host cell genome. A pre-defined genomic location may also herein be referred to as a “safe harbor site” and/or as a “recombination site”. At the pre-defined genomic location of the host cell, the recombination event between nucleic acid sequence I1 and I2 facilitated by the presence of the first DNA enzyme will occur, initiating expression of the first selection marker and indicating a successful integration event. Basically, the pre-defined genomic location comprises a nucleic acid sequence comprising a recognition site for a first DNA enzyme, a nucleic acid sequence comprising a recognition site for a second DNA enzyme and a promoter nucleic acid sequence.

Herein, when “targeted integration” is referred to, it is intended to mean the integration or the introduction of a nucleic acid sequence element or component into another nucleic acid element or component facilitating a recombination event between such sequences thereby generating a hybrid sequence from the original sequences. Such an integration event is triggered by the presence of an enzyme recognizing nucleic acid sequences in any one or several of the nucleic acid sequence elements or components forming the basis for the recombination.

A “recognition site for an enzyme” refers to a specific combination of nucleotides in a nucleic acid sequence which combination is recognised by a particular enzyme facilitating the binding of the enzyme thereto and wherein the enzyme will thereafter initiate an action at the recognition site, such as a recombination event between two sequences.

The term “DNA enzyme” referred to herein, is defined as an enzyme that acts on DNA, such as cutting pieces of DNA or cutting and integrating DNA into another DNA sequence. The term includes enzymes such as Crisps/Cas9, recombinases, integrases, nucleases etc., but the present disclosure is not limited thereto.

A “first DNA enzyme” referred to herein can be defined functionally as an enzyme that is responsible, in a method disclosed herein, for integration of the donor vector at the pre-defined genomic location of the host cell. The function of the first DNA enzyme is to introduce, not remove, nucleic acid sequences into the pre-defined genomic region. The first DNA enzyme can be one specific enzyme, or it can be different enzymes, when used in a method disclosed herein. This is e.g. if the integration of the donor vector is sequential, and thereby repeated multiple times, introducing multiple copies/variants of the nucleic acid sequence of interest/donor vector into the pre-defined genomic location of the host cell, or if a reversible integration of nucleic acid sequences of interest is performed. Examples of “first DNA enzymes” for use in the context of the present method are given elsewhere herein.

A “second DNA enzyme” referred to herein can be defined functionally as an enzyme that is responsible, in a method disclosed herein, for excision of a nucleic acid sequence region from the pre-defined genomic location having integrated a donor vector, wherein said nucleic acid sequence region is flanked by specific sequences recognized by the second DNA enzyme. When the second DNA enzyme recognises the sequences, it will cut out the nucleic acid sequence component in between these sequences. Examples of “second DNA enzymes” for use in the context of the present method are given elsewhere herein.

“In the presence of a first DNA enzyme” and/or “in the presence of a second DNA enzyme” means that a first and/or a second DNA enzyme is provided in any form as described herein, e.g. as a protein, expressed from a donor vector, a separate expression vector, an expression cassette present in the genome of a cell, a synthetic mRNA etc. “In the presence” is intended to refer to that the function of the first DNA enzyme and/or the second DNA enzyme is provided in any suitable way disclosed herein.

A “selection marker” referred to herein, is a marker that can indicate that a specific event has occurred, such as e.g. in the present context, that an integration of a donor vector has occurred at the pre-defined genomic location of the host cell (first selection marker). The selection marker is often a fluorescent protein that will be expressed by the host cell once the donor vector has been integrated at the correct site of the host cell genome. The expression of the fluorescent protein can e.g. be detected by FACS (Fluorescence-activated cell sorting). Other possible selection markers are mentioned elsewhere herein.

A “first selection marker”, also abbreviated “SM1” herein, can be defined as a silent, non-active or promoter-less selection marker when present in the donor vector. The first selection marker contains a non-coding stretch that is compatible with a promoter that is present in the pre-defined genomic location. Once the donor vector has been integrated into the correct position in the pre-defined genomic position, the first selection marker can be expressed as it now has a promoter to initiate the transcription. Once the selection marker is expressed, the cell population expressing the first selection marker can be selected as positive for stable integration of the donor vector at the pre-defined genomic position. The first selection marker can also be referred to as a “reporter” herein. Examples of suitable first selection markers are provided elsewhere herein.

A “second selection marker”, also abbreviated “SM2” herein, which is an optional feature of a donor vector disclosed herein, can be defined as a non-silent, active and/or functional selection marker when present in the donor vector. The selection marker is encoded as part of an expression cassette, i.e. the selection marker will be expressed transiently upon entry into the cell and later promote stable expression independently of where in the genome it is introduced. The second selection marker is in most aspects of the method presented herein a negative selection marker meaning that cells expressing this marker is preferably not used for recombinant protein production as these cells have (also) integrated a donor vector elsewhere than at the pre-defined genomic position.

### DETAILED DESCRIPTION

There is provided herein a method utilizing a specific Site-Directed Integration (SDI) system for targeted and detectable integration of a donor vector into pre-defined genomic locations of isolated eukaryotic cells. In addition, the method allows for the identification of random integration events of donor vectors into other parts of the eukaryotic host cell genome than into the pre-defined location.

This in combination provides for the “double” selection of populations of cells having positively integrated a donor vector at the target site (pre-defined genomic location) of the host cell genome, preferably in the absence of additional random integration of donor vectors at other positions of the host cell genome thereby providing an optimized system for subsequent recombinant protein expression. The method uses a combined selection strategy based on a positive (integration at pre-defined genomic location) and a subsequent negative (absence of a random integration event) selection of a population of cells.

The total solution is based on the integration of so-called “Landing Pad” (LP) sequences at pre-defined genomic locations selected for their ability to support high transcription and long-term stability of the same. The Landing pads are designed together with matching donor vectors enabling controlled integration into the pre-defined sites and straight-forward selection of cells in which only the desired integration has occurred. Herein, pre-defined genomic locations and Landing pad/Landing pad sequences may be used interchangeably.

The basic design of the SDI system uses a combination of two classes of DNA enzyme recognition sequences together with two different DNA enzymes, for example specific recombinases to enable (i) Integration of a donor vector comprising a nucleic acid sequence of interest at a pre-targeted genomic location of an eukaryotic host cell, (ii) Selection of cells having integrated a single copy of the donor vector at the pre-defined genomic location using at least one, or possibly two, orthogonal selection steps and (iii) Optionally removal of undesirable sequences from the donor vector at the pre-defined genomic location.

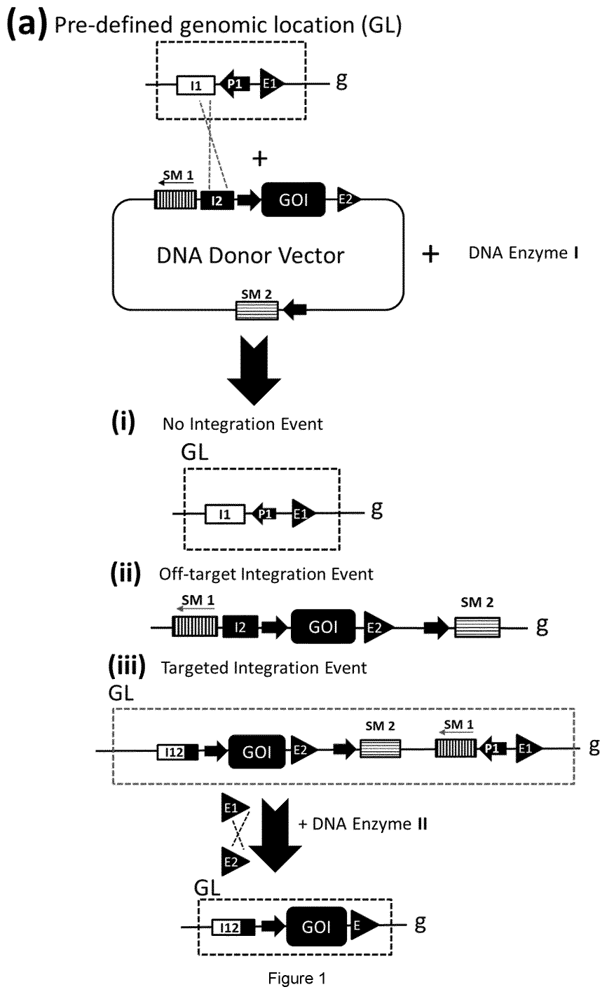

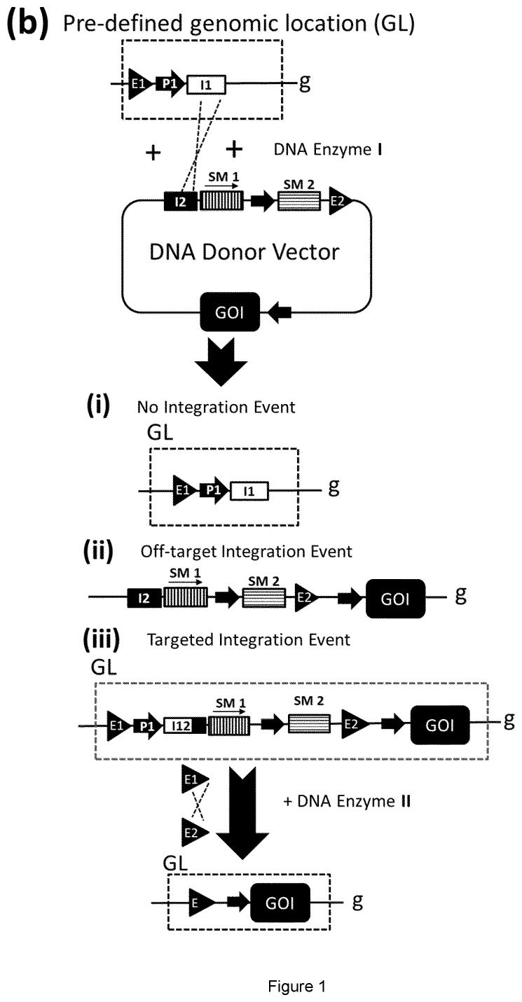

The general implementation of the method is outlined in FIG. 1. The pre-defined genomic location of said isolated eukaryotic cell comprises (i) a nucleic acid sequence I1 comprising a recognition site for a first DNA enzyme, (ii) a nucleic acid sequence E1 comprising a recognition site for a second DNA enzyme and (iii) a promotor nucleic acid sequence P1 comprising a start transcription site. I1, E2 and P1 are configured in either of the two symmetric 5′-3′ sequence orientations O1=[I1, P1 with 3′-5′ directionality, E1] or O2=[E1, P1 with 5′-3′ directionality, I1]. The donor vector comprises (i) a nucleic acid sequence I2 promoting recombination with I1 in the presence of said first DNA enzyme, (ii) a first selection marker gene (SM1) lacking a promotor, (iii) a recognition site E2 for said second DNA enzyme, (iv) an Integration Cassette IC and optionally (v) an active expression cassette for a second selection marker gene (SM2). SM1, SM2 (when present), E2 and IC are configured in either of the two symmetric clockwise orientations O3=[I2, IC, E2, SM2, SM1 with anti-clockwise directionality] or O4=[I2, SM1 with clockwise directionality, SM2, E2, IC]. Nucleic acid sequence elements present in the pre-defined genomic location and the Donor vector are always configured in either of the two matching orientations (a) O1/O3 or (b) O2/O4.

Integration of the full donor vector or parts of the donor vector into the pre-defined genomic location of said isolated eukaryotic cell is achieved by introducing the donor vector into the cell in the presence of a first DNA enzyme, wherein the presence of the first DNA enzyme enables recombination between the nucleic acid sequence I2 of the Donor vector and the nucleic acid sequence I1 present at the pre-defined genomic location of the cell.

Integration at the pre-defined genomic location positions the SM1 gene so that P1 can achieve transcription of the SM1 gene and hence expression of the SM1 gene product. Accordingly, cells having integrated the full donor vector or parts of the donor vector at the pre-defined genomic location can be selected and isolated by using expression of SM1 as a criterion for positive selection.

Optionally, undesirable sequences that can potentially negatively impact the intended functionality of isolated cells can be specifically removed from the pre-defined genomic location in a complementing step leaving only the Integration Cassette (IC) and residual sequences from I1, I2, E1 and E2.

Upon integration of the full donor vector or parts of the donor vector at the pre-defined genomic location, plasmid backbone sequences (i.e. sequences for plasmid propagation in bacteria) as well as expression cassettes for SM1 and SM2 (if present) becomes flanked by the two nucleic acid sequences E1 and E2 (see FIG. 1). In the presence of said second DNA enzyme this sequence region flanked by E1 and E2 is excised from the pre-defined genomic location via the second DNA enzyme acting on E1 and E2. Cells having excised the region flanked by E1 and E2 can be selected and isolated in a negative selection step based on the absence of SM1 expression (if SM2 is not present in the original donor vector) and/or the absence of SM2 expression (if SM2 present in the original donor vector). Besides achieving removal of undesired sequences, this complementing selection step always increases the specificity in isolation of cells having integrated the full donor vector or parts of the donor vector at the pre-defined genomic location as any cell having achieved activation of SM1 (through non-specific mechanisms) after integration outside the pre-defined genomic location will not have SM1 flanked by E1 and E2 and hence will not be selected in a negative selection step based on SM1 expression.

With SM2 present in the donor vector a selection step with improved functionality can be performed following the action of said second DNA enzyme. As SM2 is provided as an active expression cassette, any copy of the donor vector integrated at an undesired genomic location will result in expression of SM2. Importantly however, such integration events will not lead to the SM2 expression cassette being flanked by E1 and E2 as E1 is only present at the pre-defined genomic location. Hence, following the action of said second DNA enzyme leading to excision of sequence regions flanked by E1 and E2, cells having integrated a single copy of the Integration Cassette (IC) at and only at the pre-defined genomic location can be selected and isolated in a negative selection step based on the absence of SM2 expression.

The Integration Cassette (IC) typically comprises an expression cassette for a Gene of Interest (GOI) but applications of the method are not limited thereto.

Specific implementations and further examples of the general method will generally be exemplified using only one of the two possible symmetric orientations of key sequence elements present at the pre-defined genomic location and in the donor vector but is not limited thereto.

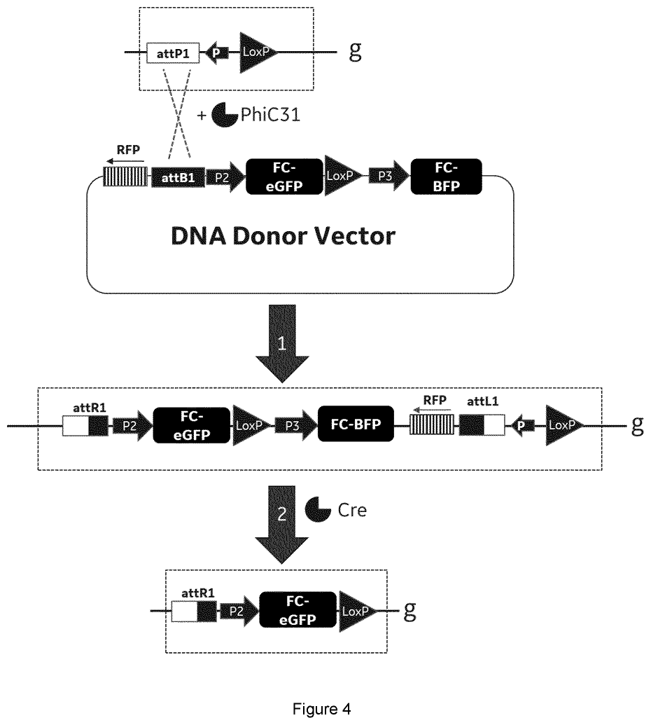

One specific implementation of the design concept is outlined in FIG. 4 featuring an Landing Pad (LP1P1) and a DNA Donor Vector. The results of the experiment performed based on the implementation is also further illustrated and discussed in the experimental section in Example 2. This implementation is merely an example of one way of performing the invention, but it is not intended to be limited thereto.

Accordingly, in one implementation as illustrated in FIG. 4, the eukaryotic host cell line contains in the pre-defined genomic location a first recombinase recognition sequence (attP1) for the recombinase PhiC31 recombinase, a promotor in 3′ to 5′ orientation and a second recombinase recognition sequence (loxP) for the recombinase Cre recombinase.

PhiC31 recombinase is a DNA recombinase derived from Streptomyces phage φC31. This enzyme can mediate recombination between two nucleic acid sequences attB and attP. Cre recombinase is also a site-specific recombinase which is used in the present system to subsequently excise the selection system and the plasmid bacterial backbone. Accordingly, the Cre recombinase can be described as “cleaning up” the vector backbone from non-useful sequences once the initial selection has been made. Both PhiC31 recombinase and Cre recombinase are well-known enzymes used in Site Specific Recombination ([10]).

The matching DNA donor vector includes a first selection marker lacking a promoter (here exemplified by RFP, Red Fluorescent Protein) encoded in anticlockwise orientation, a matching PhiC31 recombinase recognition sequence (attB1), expression cassette(s) comprising a nucleic acid sequence encoding a protein of interest, a complementing recombinase recognition sequence (loxP) for the Cre recombinase, a fully functional expression cassette for a second selection marker (optional, here exemplified by FC-eGFP) and a plasmid backbone (containing sequences for bacterial propagation etc.).

Co-transfecting the DNA Donor Vector and a vector for expression of PhiC31 into an eukaryotic host cell comprising a pre-defined genomic location of a Landing Pad (LP) sequence, will lead to integration of the donor vector at the LP via PhiC31 mediated recombination of attP1 and attB2 for a fraction of the transfected cells. Upon integration at the pre-defined genomic location, the promoter-less selection marker will be positioned so that it is activated by the promotor in the pre-defined genomic position. Activity of the first selection marker can then be used to select for cells having undergone integration at the LP (using FACS in the case of RFP). Proper selection should generate a pool of cells where most cells have a single copy integrated at the LP. However, a fraction of the cells is expected to have additional copies integrated via off-target integration mechanisms, such as DNA repair mediated random integration and PhiC31 mediated integration at genomic pseudo-attP sequences. To select against such events and at the same time enable removal of selection marker cassettes and plasmid backbone at the pre-defined genomic location (i.e. a “cleaning up”) a second recombinase mediated step has been designed.

Since the pre-defined genomic location contains a loxP sequence and the DNA Donor Vector also contains a strategically placed loxP sequence, integration events at the pre-defined genomic location will contain both selection markers (as well as other unwanted sequence elements such as the plasmid backbone), flanked by two loxP sequences. In contrast, most off-target events should not lead to loxP flanked selection markers (some random integration events of concatemerized donor vectors could lead to flanked second selection marker genes, but this should be extremely rare). By using a second transfection of a vector encoding the Cre recombinase, the region being flanked by loxP sequences can be excised from the genome of corresponding cells. Cells having a single copy integrated at the pre-defined genomic location (lacking off-target integration), as well as having unwanted sequence elements removed, can hence be selected for via the absence of selection marker activity (absence of eGFP activity using FACS). This is also called selection by negative selection.

Some key common theoretical benefits of the general SDI system disclosed herein are: (1) Enables selection steps minimizing the likelihood that an isolated cell differs from the desired outcome of having a single copy of i.e. a Gene of interest (GOI) integrated at and only at the pre-defined genomic location. This is important in CLD campaigns as it reduces biological variation and hence screening needs. It also improves the likelihood that isolated cells from a CLD campaign will behave well in a platform culture process. For optimization of expression cassette designs based on transfection of a Donor vector mixture comprising a library of expression cassette designs this is a critical feature as there need to be a one to one correlation between a cellular phenotype and a single corresponding gene cassette design.

(2) Only the sequence which contributes to the productivity of the cell line is retained. No cellular resources are wasted on expression of selection marker protein or on expression of truncated GOI versions, as can be the case for Random Integration (RI) based Cell Line Development (CLD). Presence of sequences of bacterial origin with a potential negative impact on long term expression stability of a GOI can be avoided.

(3) There is a flexibility in the choice of selection marker, as the selection marker is not part of the pre-defined genomic location sequence. Optimal selection markers can be selected based on the application.

(4) The desired integration event activates expression of the first selection marker allowing positive selection of cells with integration at the pre-defined genomic location with high specificity. Using selection markers such as fluorescent proteins or cell surface markers this enables very short time periods between transfection and selection of positive integrants (using e.g. FACS or MACS). Two to three days should be possible to obtain a result. This shortens the time needed for i.e. a CLD campaign. In addition, early isolation of cells having undergone integration at the desired location from cells having undergone undesired integration events or no integration can have further benefits as it minimizes the risk of desired cells being outgrown by undesired cells. Hence efficiency and performance of the method can be improved compared to methods lacking this feature.

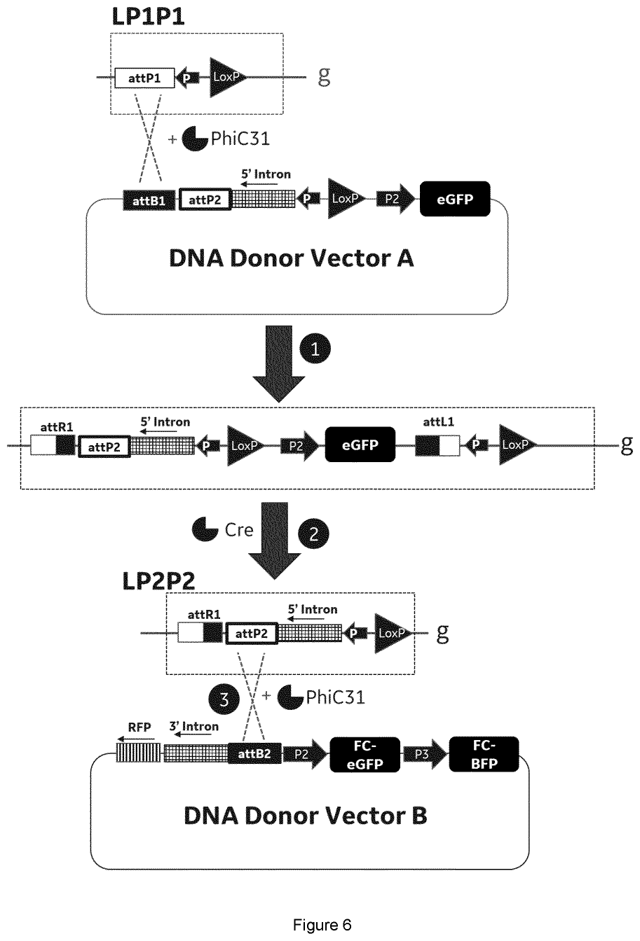

(5) The method allows for sequential integration at the same genomic location without build-up of unwanted sequence. This can be achieved by placing new sequences needed for a second integration event at the pre-defined genomic location downstream of the first GOI (or nucleic acid sequence of interest) as illustrated for PhiC31 used as a first DNA enzyme in FIG. 6. This feature would also enable generation of host eukaryotic Cell Lines with multiple Landing Pads that can be individually addressed. This enables multiple copies of a GOI to be integrated at one or several pre-defined genomic locations to achieve increased expression of the corresponding protein of interest (POI). Alternatively, it can be utilized to enable protein and clone specific Cell Line engineering via controlled integration of cellular effector proteins improving expression.

Preferred implementations of the method utilizing serine recombinases such as PhiC31 or Bxb1 as a first DNA enzyme in combination with a single matching recombinase recognition sequence pair (i.e. attP/attB) further have the potential for superior integration efficiencies. Specifically so in combination with said promoter P1 or P2 functionally fused to the 5′-part of a split intron. PhiC31 or Bxb1 mediated recombination of their corresponding attP/attB pairs are irreversible reactions and hence in theory integration should be limited only by transfection efficiency and plasmid stability. This is in contrast with Cre based integration or CRISPR/Cas9 based integration where competing non-productive reaction paths may exist.

Hence, the present disclosure provides for a novel and improved way of an efficient and selective targeted integration of nucleic acid sequences of interest (for example encoding proteins of interest) into host cells. An isolated host cell having selectively integrated a single copy of a donor vector comprising nucleic acid sequences of interest will present an excellent system for recombinant protein production which will find use in many different application areas.

Accordingly, in a first aspect, the present disclosure relates to a method for targeted integration of a donor vector into a pre-defined genomic location of an isolated eukaryotic cell, said method comprising:

i) Providing an isolated eukaryotic cell comprising a pre-defined genomic location, which pre-defined location comprises:

a. nucleic acid sequence I1 comprising a recognition site for a first DNA enzyme;

b. a nucleic acid sequence E1 comprising a recognition site for a second DNA enzyme; and

c. a promotor nucleic acid sequence P1;

ii) Providing a donor vector comprising:

a. a nucleic acid sequence I2;

b. a nucleic acid sequence of interest;

c. a nucleic acid sequence E2 comprising a recognition site for said second DNA enzyme;

d. a nucleic acid sequence encoding a first selection marker; e. optionally an expression cassette encoding a second selection marker;

iii) Contacting the donor vector with the cell in the presence of a first DNA enzyme, wherein the presence of the first DNA enzyme enables recombination between the nucleic acid sequence I2 of the donor vector and the nucleic acid sequence I1 present in the pre-defined genomic location of the cell;

iv) Selecting a cell having the donor vector integrated at the pre-defined genomic location by detecting the expression of the first selection marker in the cell, wherein the expression of the first selection marker is activated by the promotor nucleic acid sequence P1 at the pre-defined genomic location; and

v) Isolating the cell selected in the preceding step.

The first selection marker may also be abbreviated and referred to as “SM1” herein.

The second selection marker may also be abbreviated and referred to as “SM2” herein.

Non-limiting examples of first DNA enzymes are DNA recombinases, such as a PhiC31 or Bxb1 recombinase, and as described elsewhere herein. A characterizing feature of a recombinase when used as a first DNA enzyme is that it will introduce, not remove, nucleic acid sequence regions into the pre-defined genomic region.

Non-limiting examples of the second DNA enzyme are DNA recombinases, such as PhiC31 recombinase, Bxb1 recombinase, Cre recombinase and Dre recombinase, and as described elsewhere herein. A characterizing feature of a recombinase when used as a second DNA enzyme is that it will remove, not introduce, nucleic acid sequence regions from the pre-defined genomic region.

A nucleic acid sequence I1 comprising a recognition site for a first DNA enzyme may be an attP or an attB site for a PhiC31 or Bxb1 recombinase present in said pre-defined genomic location, or as otherwise exemplified herein depending on which first DNA enzyme is being used in the present context. As an example, it can also be a loxP site for a Cre recombinase, or as otherwise exemplified herein.

A nucleic acid sequence I2 can be an attB site or an attP site (recognition site) for a PhiC31 or Bxb1 recombinase present in said donor vector, or as otherwise exemplified herein depending on which first DNA enzyme is used in the present context. As an example, it can also be a loxP site for a Cre recombinase, or as otherwise exemplified herein.

A nucleic acid sequence E1 can be a loxP site for a Cre recombinase or a roxP site for a Dre recombinase. It can also be an attP or an attB site for a PhiC31 or Bxb1 recombinase, or as otherwise exemplified herein.

A nucleic acid sequence E2 can be a loxP site for a Cre recombinase or a roxP site for a Dre recombinase. It can also be an attP or an attB site for a PhiC31 or Bxb1 recombinase, or as otherwise exemplified herein.

If the first DNA enzyme is a PhiC31 recombinase, the second DNA enzyme is not a PhiC31 recombinase. The same applies to any other first and second DNA enzymes, i.e. the first and second DNA enzymes are never identical in the same SDI system.

Herein, the first selection marker (SM1) of said donor vector may be linked to a gene coding for said second DNA Enzyme via an IRES element or the amino acid sequences of SM1 and

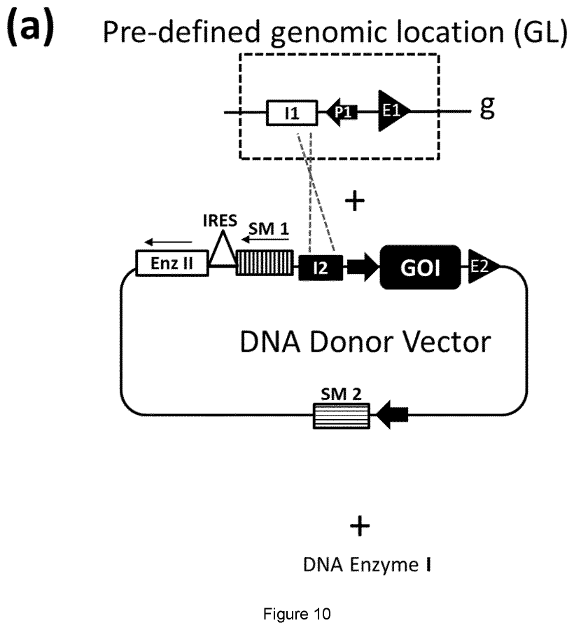

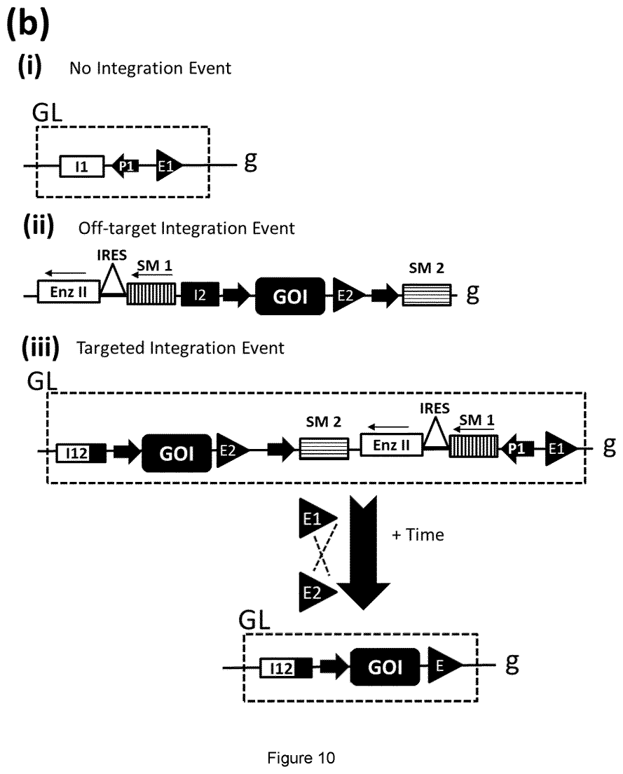

Said second DNA enzyme fused by a self-cleaving peptide such that both the first selection marker and the second DNA Enzyme is activated upon integration at the pre-defined genomic location. This is illustrated in FIG. 10. This ensures presence of the second DNA enzyme once the donor vector has been integrated into the pre-defined genomic location and no further introduction of nucleic acid vectors are needed to proceed with the steps of the method. Expression of SM1 can proceed until the intra-cellular concentration of the second DNA enzyme has reached a high enough value to promote nuclear localization and excision of the sequence region flanked by E1 and E2. By proper timing of the positive selection step, cells having undergone integration at the pre-defined genomic location will contain levels of SM1 allowing positive selection.

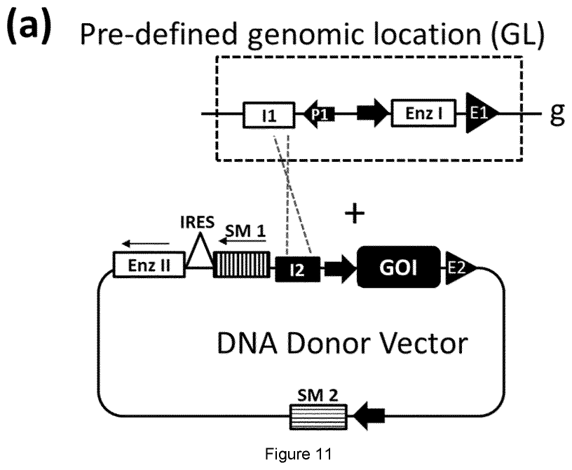

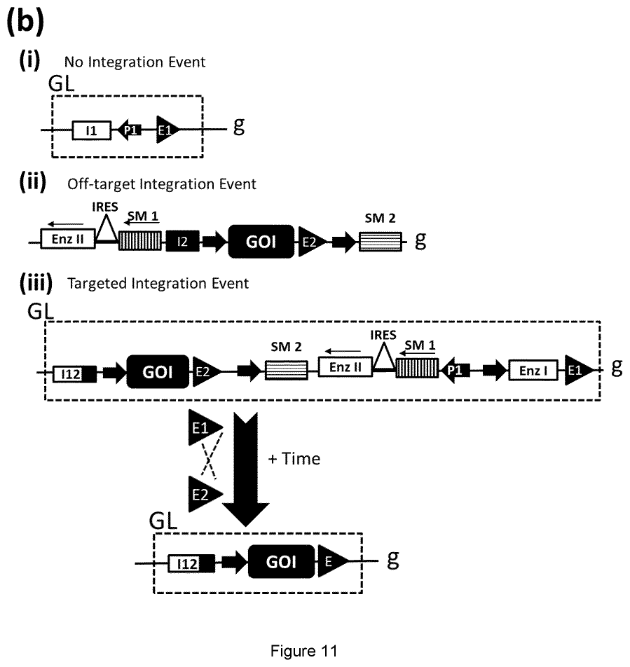

Herein, the pre-defined genomic location may also comprise an expression cassette for said first DNA Enzyme, located so that upon integration of the donor vector at the pre-defined genomic location it becomes flanked by recognition sites for said second DNA Enzyme and excised from the pre-defined genomic region via the action of said second DNA Enzyme. This is illustrated in FIG. 11. This further simplifies the method and should improve the likelihood of high integration efficiencies. Since the expression cassette is removed during later steps of the method, no cellular resources are wasted on expression of the first DNA enzyme in the final isolated cell and any negative consequences of long-term presence of the first DNA enzyme are avoided.

Accordingly, the first DNA enzyme may be provided by expression from the pre-defined genomic location or by introduction into the cell in any form yielding transient presence of said first DNA enzyme in said cell. This includes introduction of an isolated protein per se, the introduction of a separate expression plasmid comprising an expression cassette for said first DNA enzyme, the presence of an active expression cassette for said first DNA enzyme in said donor vector or introduction of a synthetic mRNA encoding said first DNA enzyme.

As previously mentioned herein, all aspects of the present disclosure allow flexibility in the choice of selection markers without having to make any changes to the pre-defined genomic location. SM1 can be selected from the groups of (i) antibiotic resistance genes, (ii) metabolic enzyme genes such as GS or DHFR, (iii) Fluorescent Protein genes or (iv) Cell surface markers such as CD4 or CD10. SM2 can be selected from the groups of (i) Toxic product generating enzymes such as TK, (ii) Fluorescent Protein genes or (iii) Cell surface markers such as CD4 or CD10.

Preferably both selection markers are selected from the groups of (i) Fluorescent protein genes or (ii) Cell surface markers allowing fast selection steps via methods such as FACS or MACS.

The expression of the first or second selection marker can be detected e.g. by using FACS, if the selection marker is a Fluorescent protein. If the selection marker is an antibiotic resistance gene, the integration can be detected by culturing cells in the presence of the corresponding antibiotic. If the cells survive in a media to which an antibiotic has been added, the donor vector has been successfully integrated.

There is also provided herein a method, further comprising a step vi), comprising excising a nucleic acid sequence flanked by the nucleic acid sequences E1 and E2 from the pre-defined genomic location of the cell isolated in step v) of a method herein in the presence of a second DNA enzyme, wherein the presence of the second DNA enzyme enables recombination between the nucleic acid sequences E1 and E2, wherein the presence of a nucleic acid sequence flanked by the nucleic acid sequences E1 and E2 in said cell is indicative of a stable integration of the donor vector into the pre-defined genomic location of the cell.

As mentioned herein, the recombinases are useful for excising nucleic acid sequences flanked by the appropriate nucleic acid regions (E1 and E2) in the host cell genome. This is a step mainly to “tidy up” in the host cell genome as some parts of the nucleic acid sequence introduced into the pre-defined genomic location will be superfluous once an integration and selection has been made. Their presence may also consume cell energy. Excising of a nucleic acid sequence means that the second DNA enzyme by binding to specific combinations of nucleotides, i.e. E1 and E2, is capable of cutting and removing nucleic acid sequence parts from the host cell genome. The presence of the nucleic acid sequences E1 and E2 at the pre-defined genomic location is in principle proof of that a stable integration of the donor vector has occurred.

Herein, step vi) may form part of step iii), or may be performed after step iii), such as after step iv) or after step v) of said method. Step vi) may also be performed before step v).

There is also provided a method, wherein the donor vector of step ii) further comprises e) an expression cassette encoding a second selection marker and wherein the cell isolated in step v) additionally has been selected based on its non-expression of the second selection marker, wherein expression of the second selection marker signals that a donor vector has been integrated at a different location than the pre-defined genomic location of a cell.

There is also provided a method herein, wherein the donor vector of step ii) further comprises e) an expression cassette encoding a second selection marker, and wherein the cell isolated in step vii) has been selected based on its non-expression of the second selection marker, wherein expression of the second selection marker signals that a donor vector has been integrated at a different location than the pre-defined genomic location of a cell.

Said expression cassette encoding a second selection marker is positioned in said donor vector, so that upon integration at the pre-defined genomic location it becomes flanked by E1 and E2 sequences. However, if the donor vector is integrated outside the pre-defined genomic location, said expression cassette encoding a second selection marker will not be flanked by E1 and E2.

Accordingly, if it is possible to detect, e.g. by FACS, expression of a second selection marker (SM2) among cells in the cell population following action of the second DNA enzyme, this means that an undesired integration event of a donor vector has occurred at another position in the cell. Such cells could be removed to select for (by negative selection) the cells where integration of the donor vector has occurred only at the pre-defined genomic location.

There is also provided a method wherein step vi) is performed after step v), and the method further comprises a step vii), performed after step vi), comprising isolating a cell in which the nucleic acid sequence being flanked by the nucleic acid sequences E1 and E2 has been excised from the pre-defined genomic location of the cell isolated in step vi).

The second DNA enzyme may be provided as an isolated protein per se, it may be expressed from an expression cassette on a separate expression vector or plasmid or may be expressed from a synthetic mRNA encoding said second DNA enzyme. It may also be expressed from the donor vector once integrated into the pre-defined genomic location as previously described.

There is also provided a method herein, wherein the nucleic acid sequence of interest of the donor vector of step ii) comprises at least one expression cassette comprising a gene encoding a protein of interest. The protein of interest can be any type of recombinant protein that the user wishes to express, such as antibodies or other therapeutic proteins.

There is also a provided a method herein wherein the excised nucleic acid sequence lacks the at least one expression cassette containing a gene encoding a protein of interest.

Cell Line Development based on Random Integration (RI) typically results in top clones having multiple copies of the target gene(s) integrated. To ensure that the method of the present disclosure also can generate the transcription levels (# of mRNA copies per cell) needed for competitive protein expression levels it would be advantageous to have the ability to increase the target gene copy number in a controlled way. Integration of multiple copies in one go may be problematic as the size of the expression plasmids becomes an issue for bacterial expansion efficiency, transfection efficiency, plasmid stability in CHO cells and integration efficiency.

Therefore, there is also provided herein a method for sequential integration of multiple copies of a nucleic acid of interest into a host cell genome. Besides reducing expression plasmid size, inserting copies in a sequential manner offers the potential added feature of gradually increasing the recombinant expression load put on cells. This in turn could improve the likelihood of isolating high-producing phenotypes due to the possibility of gradual adaptation to a new stressful environment. As previously referred to, repeated integration at the pre-defined genomic location can also be utilized to enable protein and clone specific Cell Line engineering by first introducing expression cassette(s) for a protein of interest followed by introduction of cellular effector genes that can improve the expression of the protein of interest in subsequent integration steps.

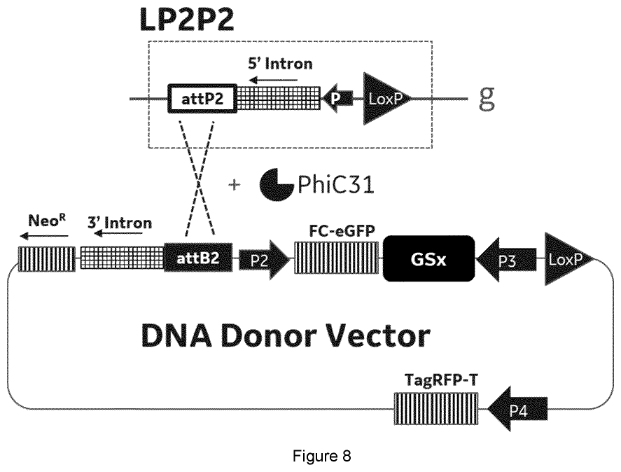

A key feature of the PhiC31/att based technology presented herein holding the key to straightforward and controlled sequential integration of multiple copies is the presence of orthogonal attP/attB pairs. Orthogonal attP/attB pairs differ from the native sequences only at the central nucleotide pair. An example of repeated integration at the same genomic location using orthogonal recognition sites for the first DNA enzyme is shown in Example 3 of the Experimental section, and in FIGS. 6 and 8. Of course the same approach as the PhiC31/att based technology described herein can be used for other DNA enzymes, such as Cre, Dre, Flp or CRISPR/Cas9 for which orthogonal DNA enzyme recognition pairs/sequences exists.

Accordingly, to provide for sequential integration of multiple nucleic acid sequences of interest, there is also provided herein a method comprising steps as defined elsewhere herein, wherein the donor vector of step ii) of a method presented herein further comprises: f. a nucleic acid sequence I3 comprising a recognition site for a first DNA enzyme; and g. a promotor nucleic acid sequence P2.

The presence of a further recognition site I3 in said donor vector provides for the repeated targeted integration of two or more donor vectors at the same pre-defined genetic location, as explained in the above. Once the first recombination event has occurred within the nucleic acid sequence pair I1/I2 (e.g. attB1/attP1), generating a hybrid sequence (attR1), there remains a further recognition site I3 (e.g. attP2) that can facilitate for the next round of integration with a second donor vector comprising a further recognition site I4 (e.g. attB2). FIG. 6 illustrates how such a sequential integration procedure may be performed. The method comprises rounds of excision of nucleic acid sequence components from the pre-defined genomic location having integrated a first or further donor vector(s) to make room for integration of additional nucleic acid sequences of interest.

Accordingly, there is provided herein a method for sequential targeted integration of n additional donor vectors into the pre-defined genomic location of the eukaryotic cell wherein the first donor vector further comprises, in addition to the components previously mentioned herein: f. a nucleic acid sequence I3 comprising a recognition site for a first DNA enzyme; and g. a promotor nucleic acid sequence P2.

The method for sequential targeted integration of n additional donor vectors into the pre-defined genomic location of the eukaryotic cell comprises, in addition to performing at least steps i) to iv) and optionally v), vi) and/or vii) in any suitable order, performing a sequential targeted integration of n additional donor vectors into the pre-defined genomic location of the isolated eukaryotic cell,

wherein n is an integer 1, such as 2, 3, 4, 5, 6, 7, 8, 9, 10 or any other number; the method comprising:

(A) Integrating a first additional donor vector into the pre-defined genomic location of the cell, comprising:
I. Providing the cell isolated in step v) or isolated in step vii);
II. Providing a donor vector comprising:
   A. a nucleic acid sequence I4, which in the presence of said first DNA enzyme is capable of recombining with the corresponding nucleic acid sequence I3 present in the cell provided in the preceding step;
   B. a nucleic acid sequence of interest;
   C. a nucleic acid sequence E2;
   D. a nucleic acid sequence encoding a first selection marker; and
   E. optionally an expression cassette encoding a second selection marker;
   F. optionally a nucleic acid sequence I1 comprising a recognition site for a first DNA enzyme and a promotor nucleic acid sequence P1;
III. Introducing the donor vector of step II) into the cell in the presence of a first DNA enzyme, wherein the presence of the first DNA enzyme enables recombination between the nucleic acid sequence I4 of the donor vector and the nucleic acid sequence I3 present in the pre-defined genomic location of the cell;
IV. Selecting a cell having the donor vector integrated at the pre-defined genomic location by detecting the expression of the first selection marker in the cell, wherein the expression of the first selection marker is activated by the promotor nucleic acid sequence P2 at the pre-defined genomic location of the cell;
V. Isolating the cell selected in the preceding step;
VI. Excising a nucleic acid sequence flanked by the nucleic acid sequences E1 and E2 from the pre-defined genomic location of the cell isolated in step V in the presence of a second DNA enzyme, wherein the presence of the second DNA enzyme enables recombination between the nucleic acid sequences E1 and E2, wherein the presence of a nucleic acid sequence flanked by the nucleic acid sequences E1 and E2 is in said cell is indicative of a stable integration of the donor vector into the pre-defined genomic location of the cell;
VII. Isolating a cell in which the nucleic acid sequence being flanked by the sequences E1 and E2 has been excised from said pre-defined genomic location of the cell isolated in step V;

(B) Provided that n is larger than the number of additional donor vectors integrated at the pre-defined genomic location of the cell isolated in the preceding step, integrating an additional donor vector into the pre-defined genomic location of the cell, comprising:
I. Providing the cell isolated in the preceding step, which cell comprises the nucleic acid sequence I1 integrated at the pre-defined genomic location;
II. Performing step ii), steps iii)-iv), step v), step vi), and step vii);

(C) Provided that n is larger than the number of additional donor vectors integrated at the pre-defined genomic location of the cell isolated in the preceding step, integrating an additional donor vector into the pre-defined genomic location of the cell, comprising:
I. Providing the cell obtained by performing the preceding step (B), which cell comprises the nucleic acid sequence I3 integrated at the pre-defined genomic location;
II. Repeating steps (A)II to (A)IX and step (B) until the cell has n additional donor vectors integrated at the pre-defined genomic location.

As mentioned previously herein, the recognition site I4 in the donor vector provides for recombination with the recognition site I3 already present at the pre-defined genomic location of the host cell (i.e. that was integrated in the previous round of integration). Thereby, a second, and further, copy/copies of a nucleic acid of interest can be introduced at the pre-defined genomic location by subsequent introductions of recognition site variants through the donor vector. The specific recognition site variants (pairs) used for the recombination between the introduced donor vector and the nucleic acid sequence present in the pre-defined genomic region (i.e. the first DNA enzyme recognition sites) can be re-used throughout the rounds of recombination events as long as there is a different pair of recognition sites in between each round of integration. The same is true for selection markers.

Specifically, there is provided an iterative method for integration of any desired number of donor vector copies into the pre-defined genomic location of said eukaryotic cell based on only two orthogonal pairs of recognition sequences for a first recombinase enzyme and two variants of said first selection marker wherein:

(i) Said first recombinase enzyme is selected from the group of serine recombinases such as PhiC31 or Bxb1 or mutated variants thereof;

(ii) Said two selection marker variants are selected from the groups of (a) fluorescent proteins or (b) heterologous cell surface markers;

(iii) The donor vector used in odd integration steps comprises:
(a) A first version of said first selection marker.
(b) A first recombinase recognition sequence from a first pair of recognition sequences.
(c) A first recombinase recognition sequence from a second orthogonal pair of recognition sequences;

(iv) The donor vector used in even integration steps comprises:
(a) A second version of said first selection marker.
(b) A second recombinase recognition sequence from said second orthogonal pair of recognition sequences.
(c) A second recombinase recognition sequence from a first pair of recognition sequences;

(v) Integration in odd integration steps is promoted by recombination between recombinase recognition sequences from said first pair of recombinase recognition sequences;

(vi) Integration in even integration steps is promoted by recombination between recombinase recognition sequences from said orthogonal second pair of recombinase recognition sequences;

(vii) Before integrations of odd integration steps said first version of the first selection marker is excised from the pre-defined genomic location by the presence of said second recombinase enzyme acting on recombinase recognition sequences E1 and E2;

(viii) Before iterations of even integration steps said second version of the first selection marker is excised from the pre-defined genomic location by the presence of a second recombinase enzyme acting on recombinase recognition sequences E1 and E2;

(ix) Said second recombinase enzyme is selected from the group of tyrosine recombinases such as Cre, Dre or Flp;

(x) E1=E2.

There is further provided a method wherein:
the cell of step i) comprises n pre-defined genomic locations, each of which comprises a nucleic acid sequence I, I1₁ to I1ₙ, respectively, wherein the I1₁ to I1ₙ are different from each other;
step ii) comprises providing 1 to n donor vectors, each of the 1 to n donor vectors comprising a nucleic acid sequence I2₁ to I2ₙ, respectively, which is capable of recombining with the corresponding I1₁ to I1ₙ nucleic acid sequence of said donor vector in the presence of said first DNA enzyme, and each of the 1 to n donor vectors comprising a first selection marker, SM₁ to SMₙ, wherein the SM₁ to SMₙ are different from each other;
step iv) comprises introducing the 1 to n donor vectors into the cell;
step v) comprises selecting a cell having each of the 1 to n donor vectors integrated at its corresponding pre-defined genomic location by detecting each of the different first selection markers, SM₁ to SMₙ, in the cell;

wherein n is an integer ≥2.

There is also provided a method, wherein the donor vector of step ii) further comprises:

f. a nucleic acid sequence I3 comprising a recognition site for a first DNA enzyme; and

g. a promotor nucleic acid sequence P2, and

wherein the excised sequence comprises the expression cassette containing a gene encoding protein of interest.

The fact that the excised sequence comprises the expression cassette containing a gene encoding a protein of interest means that there has been a reversible integration where the second round of recombination can introduce a new and “first” nucleic acid sequence of interest encoding a protein of interest at the pre-defined genomic location. Hence, there will not be multiple copies of the same gene of interest present in the pre-defined genomic location, which is the purpose of the sequential integration mentioned previously herein. This can be utilized for reuse of a high performing clone for the expression of another protein of interest.

In FIG. 8 and in Example 4 it is illustrated the generation of an SDI cell pool using two consecutive selection steps, i.e. where a second DNA enzyme is added after the integration to remove nucleic acid sequences that do not fulfil a purpose in the cell any longer. In this example, an antibiotic resistance gene was used as a first selection marker (SM1). A second round of selection was performed using Cre recombinase to excise nucleic acid sequences flanked by the loxP nucleic acid regions in each end. The presence of a random integration event was detected by a double positive signal of GFP/RFP (Green/Red Fluorescent Protein) using FACS. The cells were sorted based on the positive/negative GFP/RFP signal. This additional step provides for the removal of cells that may have integrated one donor vector at the pre-determined genomic location but that may also have randomly integrated a second or further donor vector(s) at a random non-target position(s) in the host cell genome.

As previously mentioned herein, said first DNA enzyme may be a recombinase. The first DNA enzyme may be a mix of different DNA enzymes, such as recombinases, as long as any of the DNA enzymes of the first DNA enzyme is not the same as the second DNA enzyme.

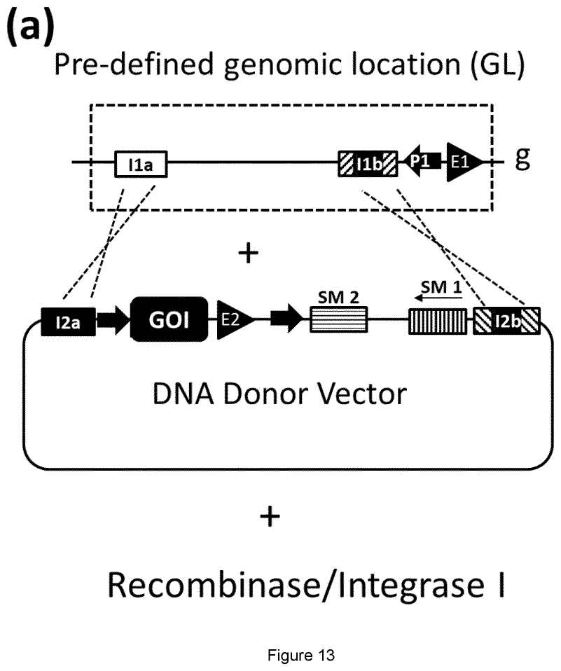

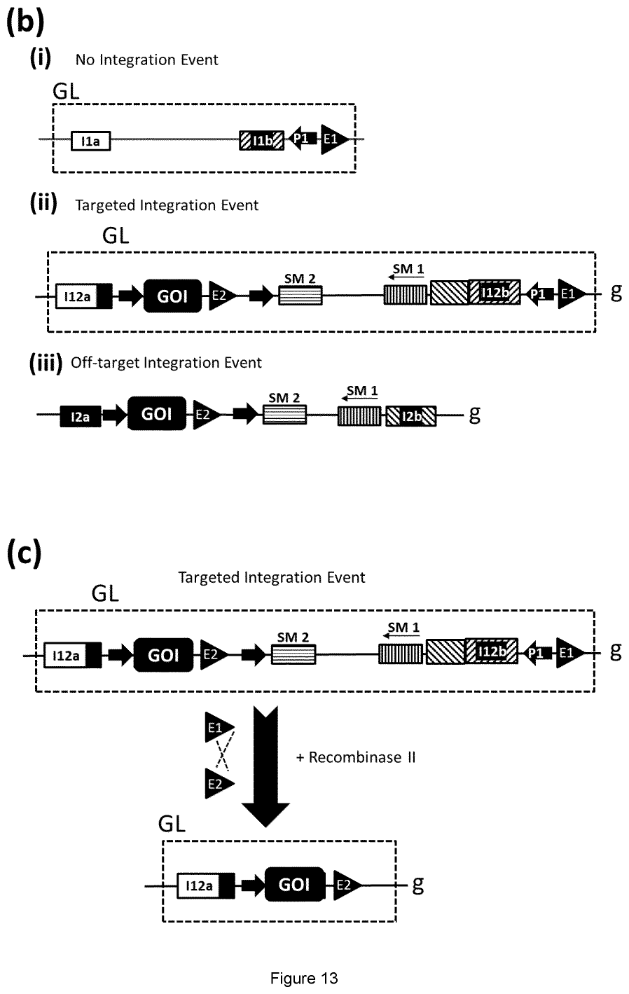

Herein, there may be more than one recognition site for said first DNA enzyme present in the donor vector, such as two or more recognition sites. This means that there will also be more than one recognition site for said first DNA enzyme present in the pre-defined genomic location, such two or more recognition sites. An example of such a system utilizing recombinases is shown in FIG. 13. FIG. 13 show a recombinase mediated cassette exchange (RMCE) to catalyze integration at the pre-defined genomic location. Variants thereof modified according to FIGS. 1-2, FIGS. 9-11 and FIG. 15 are also encompassed by the present disclosure.

Hence, in the example in FIG. 13, the pre-defined genomic location comprise in 5′-3′ sequence order; (i) a first recognition site for a first recombinase enzyme (I1a); (ii) a second recognition site for said first recombinase enzyme (I1b), (iii) a Promotor P1 with 3′-5′ directionality and (v) a recognition site E1 for a second recombinase enzyme.

In this example, the donor vector comprise in 5′-3′ sequence order; (i) a third recognition site for said first recombinase enzyme (I2a), (ii) an Integration Cassette (IC), here exemplified by an expression cassette for a Gene of Interest (GOI), (iii) a recognition site E2 for said second recombinase enzyme, (iv) an expression cassette for a second Selection Marker (SM2), (v) a gene for a first Selection Marker (SM1) encoded in 3′-5′ directionality and (vi) a fourth recognition site for said first recombinase enzyme (I2b).

Introduction of the donor vector and a first recombinase into a population of cells results in; (a) integration of the Integration cassette in the donor vector, i.e. the sequence region flanked by said third and fourth recombinase recognition sites at the pre-defined genomic location for a fraction of the cells (See FIG. 13b, panel (ii)) and (b) off-target genomic integration (outside the pre-defined genomic location) of the donor vector for a fraction of cells (See FIG. 13b, panel (iii)).

Integration at the pre-defined genomic location results in the formation of an active expression cassette for SM1 (See FIG. 13b, panel (ii)). Further, following integration at the pre-defined genomic location both SM1 and SM2 are flanked by two recognition sites for said second recombinase enzyme.

Integration by an off-target event (See FIG. 13b, panel (iii)) does not typically lead to the activation of SM1 but does integrate an active SM2 that is not flanked by two recognition sites for said second recombinase enzyme.

Cells having undergone integration at the pre-defined genomic location differs from cells with no integration event (See FIG. 13b, panel (i)) and cells having undergone only an off-target integration event through the activity of SM1. Hence, activity of SM1 can be used to select for cells having undergone integration at the LP.

To remove cells having undergone an off-target integration event in addition to integration at the pre-defined genomic location, recombinase activity of said second recombinase is introduced within cells selected for SM1 activity. For integration at the LP, this results in the excision of both SM1 and SM2 and hence their corresponding activity. For off-target integration events this reaction cannot occur and SM2 activity remains. As a result, cells having undergone only the desired targeted integration event at the pre-defined genomic location can be selected from cells having undergone a multiple integration event through absence of SM2 activity.

The pre-defined genomic location for the finally selected Cells (See FIG. 13c) does not contain the expression cassette for SM2 nor the activated expression cassette for SM1 or any residual sequence from the donor vector except a sequence created through the recombination of E1 and E2 (E).

Said first recombinase enzyme can be selected from the groups of (i) Serine recombinases or (ii) Tyrosine recombinases.

Said first to fourth recombinase recognition sites can be selected according to; (a) I1a=I1b and I2a=I2b using one matching recognition site pair for serine recombinases such as PhiC31 [I1a=I1b=attP or attB and I2a=I2b=attB or attP] or Bxb1 [I1a=I1b=Bxb1 attP or Bxb1 attB and I2a=I2b=Bxb1 attB or Bxb1 attP],

(b) Selecting two different matching recognition site pairs using mutated recognition pairs for serine recombinases such as PhiC31 (I1a and I1b=different attP variants or attB variants; I2a and I2b=different attB variants or attP variants) and

(c) I1a=I2a and I1b=I2b for tyrosine recombinases, were mutated recognition site variant pairs exist, such as Cre (LoxP1 and LoxP2 selected from available mutated loxP pairs), Dre (rox1 and rox2 selected from available mutated rox pairs) or FLP (FRT1 and FRT2 selected from available mutated FRT pairs).

Said second recombinase enzyme is different from said first recombinase enzyme and can be selected from the groups of (i) Serine recombinases or (ii) Tyrosine recombinases.

Recognition sites E1 and E2 for said second recombinase enzyme can be identical in sequence as exemplified by; (i) E1=E2=loxP or its mutated variants for use with the Cre recombinase, (ii) E1=E2=rox or its mutated variants for use with the Dre recombinase or (iii) E1=E2=FRT or its mutated variants for use with the FLP (Flippase) recombinase.

Recognition sites E1 and E2 for said second recombinase enzyme can have different sequences as exemplified by; (i) E1=attP and E2=attB or its mutated variants for use with the PhiC31 recombinase or (ii) E1=Bxb1 attP and E2=Bxb1 attB or its mutated variants for use with the Bxb1 recombinase.

The further recognition sites for the first DNA enzyme can be referred to herein as variants of I1, i.e. I1a and I1b, and variants of I2, i.e. I2a and I2b, and so on.

Accordingly, there is also provided herein a method herein, wherein

(a) I1 comprises two recombinase recognition site variants I1a and I1b; and

(b) I2 comprises two recombinase recognition site variants I2a and I2b; and

(c) I1a is capable of recombination with I2a and I1b is capable of recombination with I2b in the presence of said first DNA enzyme.

Sometimes, I1a is identical to I2a and I1b is identical to I2b. I1a, I1b, I2a and I2b may be selected from loxP, rox or FRT or variants thereof, respectively, and the first DNA enzyme may be selected from the group consisting of a Cre recombinase, a Dre recombinase and a FLP recombinase [3], respectively.

Herein, there is also provided a method, wherein:

(a) I1 comprises a single recombinase recognition site; and

(b) I2 comprises a single recombinase recognition site; and

(c) I1 and I2 are capable of recombination in the presence of said first DNA enzyme.

The recombinase recognition site comprised by I1 may also differ in sequence from the recombinase recognition site comprised by I2. The recombinase recognition sites provided herein may be selected from attB, attP, Bxb1 attP, Bxb1 attB or a variant thereof. Said recombinase may be a PhiC31 or Bxb1 recombinase or a mutant thereof.

Any variant or mutant of a recognition site/DNA enzyme will be a functionally equivalent variant or mutant thereof. The skilled person will construct and produce such a functionally equivalent variant or mutant.

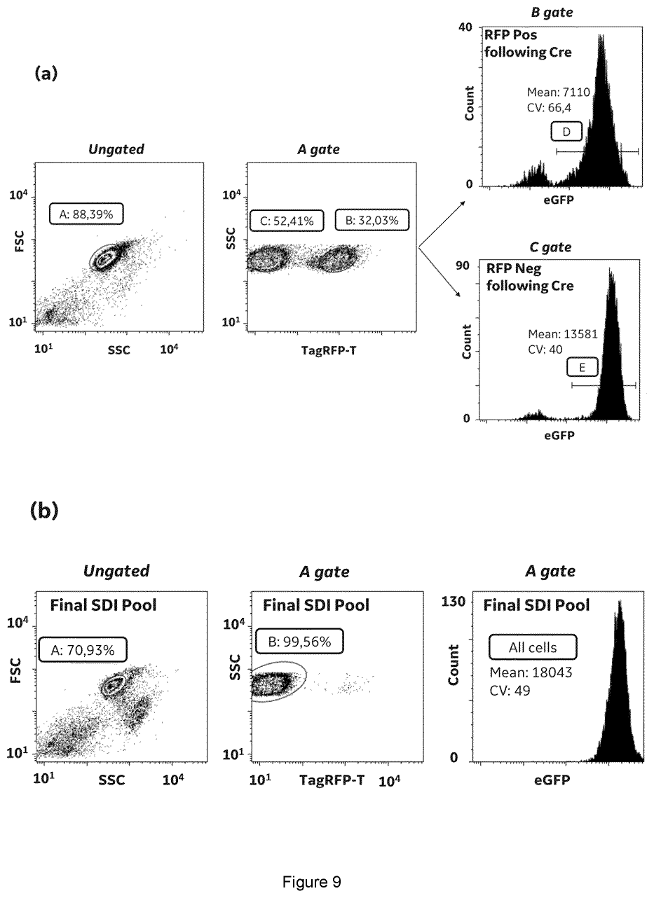

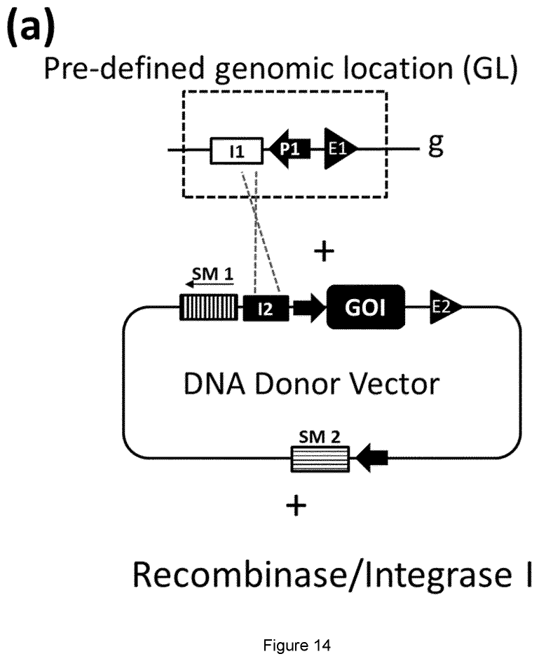

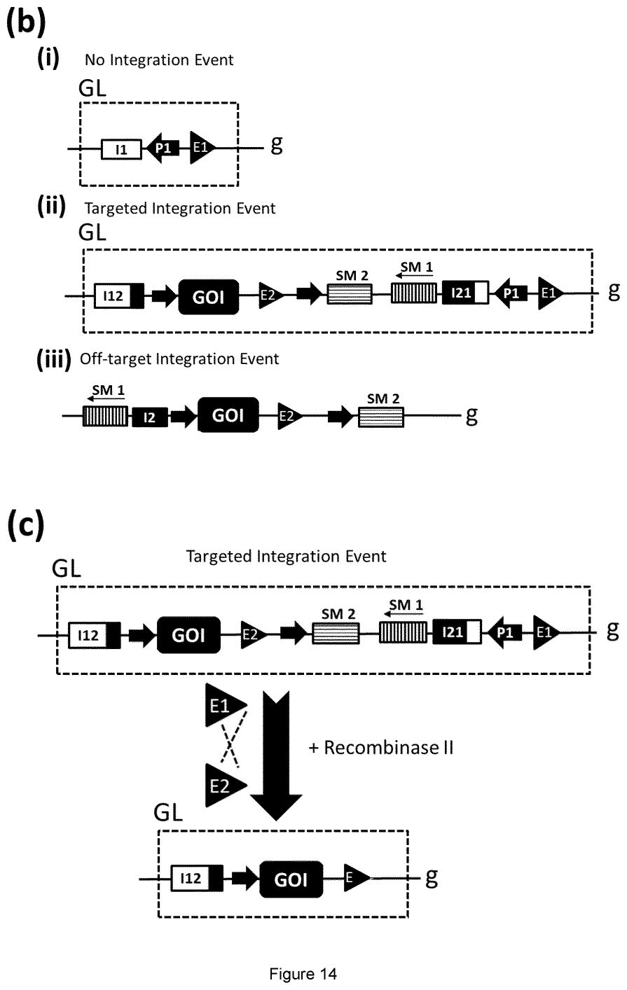

An example of using a single recombinase recognition site pair to catalyze integration at the pre-defined genomic location is shown in FIG. 14. Variants modified according to FIG. 1, FIG. 9-11 and FIG. 15 are also encompassed by this disclosure.

In this example, the pre-defined genomic location comprise in 5′-3′ sequence order; (i) a first recognition site for a first recombinase enzyme (I1); (ii) a Promotor P1 with 3′-5′ directionality and (iii) a first recognition site E1 for a second recombinase enzyme.

In this example, the donor vector comprise in 5′-3′ sequence order; (i) a second recognition site for said first recombinase enzyme (I2), (ii) an Integration Cassette (IC), here exemplified by an expression cassette for a Gene of Interest (GOI), (iii) a second recognition site E2 for said second recombinase enzyme, (iv) an expression cassette for a second Selection Marker (SM2) and (v) a gene for a first Selection Marker (SM1) encoded in 3′-5′ directionality.

Introduction of the donor vector and a first recombinase into a population of LP Cells results in; (a) integration of the donor vector at the LP for a fraction of the LP cells (See FIG. 14b, panel (ii)) and (b) off-target genomic integration (outside pre-defined genomic location) of the donor vector for a fraction of the cells (See FIG. 14b, panel (iii)).

Integration at the pre-defined genomic location results in the formation of an active expression cassette for SM1 (See FIG. 14b, panel (ii)). Further, following integration at the pre-defined genomic location both SM1 and SM2 are flanked by the two recognition sites E1 and E2 for said second recombinase enzyme.

Integration by an off-target event (See FIG. 14b, panel (iii)) does not typically lead to the activation of SM1 but does integrate an active SM2 that is not flanked by recognition sites for said second recombinase enzyme.

Cells having undergone integration at the pre-defined genomic location differs from cells with no integration event (See FIG. 14b, panel (i)) and cells having undergone only an off-target integration event through the activity of SM1. Hence, activity of SM1 can be used to select for cells having undergone integration at the pre-defined genomic location.

To remove cells having undergone an off-target integration event in addition to integration at the pre-defined genomic location, recombinase activity of said second recombinase is introduced within cells selected for SM1 activity. For integration at the pre-defined genomic location, this results in the excision of both SM1 and SM2 and hence their corresponding activity. For off-target integration events this reaction cannot occur and SM2 activity remains. As a result, cells having undergone only the desired targeted integration event at the LP can be selected from LP Cells having undergone a multiple integration event through absence of SM2 activity.

The pre-defined genomic location for the finally selected cells (See FIG. 14c) does not contain the expression cassette for SM2 nor the activated expression cassette for SM1 or any residual sequence from the donor vector except sequences created through the recombination of I1 and I2 (I12) and E1 and E2 (E).

Said first recombinase enzyme can be selected from the group of Serine recombinases such as PhiC31 and Bxb1. Said first and second recombinase recognition sites (I1 and I2) can be selected with recognition sites matching chosen recombinase according to; (a) I1=attP variant and I2=attB variant or (b) I1=attB variant and I2=attP variant.

Said second recombinase enzyme is different from said first recombinase enzyme and can be selected from the groups of (i) Serine recombinases or (ii) Tyrosine recombinases.

Recognition sites E1 and E2 for said second recombinase enzyme can be identical in sequence as exemplified by; (i) E1=E2=loxP or its mutated variants for use with the Cre recombinase, (ii) E1=E2=rox or its mutated variants for use with the Dre recombinase or (iii) E1=E2=FRT or its mutated variants for use with the FLP recombinase.

Recognition sites E1 and E2 for said second recombinase enzyme can have different sequences as exemplified by; (i) E1=attP and E2=attB or its mutated variants for use with the PhiC31 recombinase or (ii) E1=Bxb1 attP and E2=Bxb1 attB.

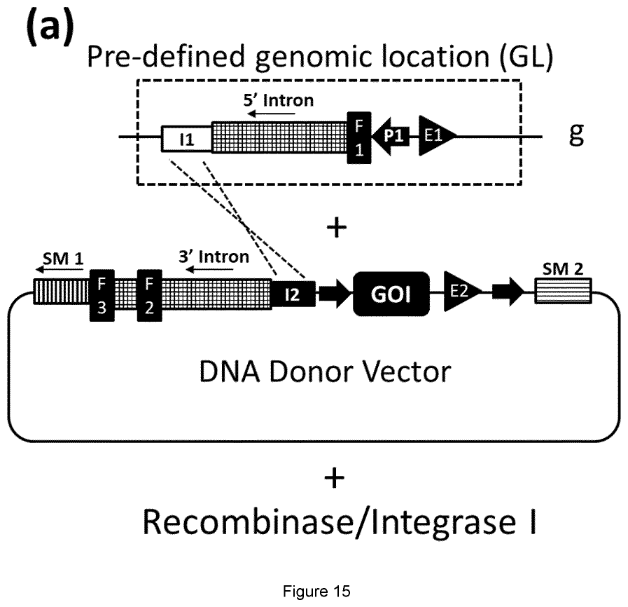

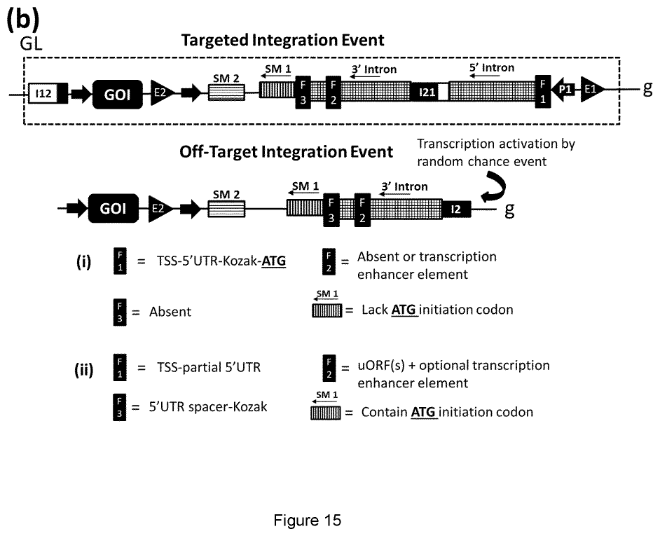

There is also provided a method, as illustrated in FIG. 15, which exemplify using a single recombinase recognition site pair to catalyze integration at the pre-defined genomic location and wherein the promotor P1 present at the pre-defined genomic location is functionally fused to the 5′-part of a split intron. Methods described earlier but modified according to FIG. 15 (i.e. using a split intron design) are also encompassed by the present disclosure.

In this example the pre-defined genomic location further comprises the 5′-part of an Intron with 3′-5′ directionality and a functional sequence region F1 with 3′-5′ directionality between said first recognition site for said first recombinase enzyme and said promotor P1 with 3′-5′ directionality.

In this example the donor vector further comprise a sequence region located between said first selection marker SM1 with 3′-5′ directionality and said second recognition site for said first recombinase enzyme. Said sequence region comprise in 5′-3′ sequence order; (a) a functional sequence region F3 with 3′-5′ directionality and (b) the 3′-part of an Intron with 3′-5′ directionality further comprising a functional sequence region F2 downstream of the splice acceptor site sequence.

Upon integration at the pre-defined genomic location (See FIG. 15b, upper panel) a complete expression cassette, including a functional Intron, for said first selection marker SM1 is formed. Hence expression of SM1 is activated.

Following an off-target integration event (See FIG. 15b, lower panel) a truncated version of the SM1 expression cassette is integrated.

By chance events transcription of the truncated SM1 cassette can occur. This can be due to (a) promotor rescue in which the donor vector is integrated in such a way that the truncated SM1 cassette becomes located in frame with a native promotor present in the cell genome or (b) cleavage and concatamerization of the donor vector such that a promotor present in the donor vector becomes re-oriented in frame with the truncated SM1 cassette followed by integration of the resulting concatamer.

Such chance events can reduce specificity in SM1 based selection of cells having integrated the donor vector at the pre-defined genomic location. By use of specific combinations of functional sequence regions F1-F3, improved specificity can be achieved (See FIG. 15b).

In a first design of F1-F3; (a) SM1 (when present in the donor vector) lacks the ATG start codon and is directly fused to the 3′-Intron, (b) F1 is made up of (from 3′-5′) a start transcription site (TSS), a first 5′-UTR region, a Kozak/translation initiation site and an ATG start codon all with 3′-5′ directionality. Following an off-target integration event this means that any SM1 gene integrated will lack a start codon and hence will not generate expression of a functional SM1 protein. However, upon integration at the pre-defined genomic location a functional expression cassette will be formed. Upon splicing of the Intron, the ATG start codon will be directly fused to SM1, leading to proper expression of SM1 protein.

In a second design of F1-F3; (a) SM1 contains an ATG start codon, (b) F3 is made up of (from 3′-5′) a second 5′-UTR region and a Kozak/translation initiation site all with 3′-5′ directionality, (c) F2 comprise at least one short upstream open reading frame (uORF) with 3′-5 directionality and (d) F1 is made up of a start transcription site (TSS) and a first 5′-UTR region. Following an off-target integration event the truncated SM1 cassette will typically retain the one or more uORFs. These uORFs will reduce initiation at the intended SM1 start codon thereby improving discrimination between off-target integration based SM1 activation and SM1 activation based on integration at the pre-defined genomic location. Preferably, multiple uORFs in series are used and placed with minimal distance to the SM1 start codon (directly downstream of the Intron Splice branch site).

The use of a split intron design also improves the expression of activated SM1 as optimal 5′-UTR sequences can be used for SM1. In designs lacking a split intron, a sequence generated through recombination of I1 and I2 (see FIG. 15) will be comprised by the SM1 5′-UTR. This leads to an extended 5′-UTR with potentially non-optimal sequence composition that can reduce the obtainable expression level of SM1 (impacting specificity in positive selection steps based on SM1). Via the use of a split intron as described herein, the I1/I2 recombination product becomes incorporated in the fully formed intron upon integration at the pre-defined genomic location (See FIG. 15). Upon generation of mature SM1 mRNA by the cell, the intron is spliced out and the corresponding SM1 5′-UTR fully defined by F1 and F2. Accordingly, the SM1 5′-UTR can be design with full control to optimize SM1 expression for an intended purpose. Variations in the design of F2-F3 further offers flexibility in the expression level of SM1 upon integration at the LP. Increasing the length of the 5′-UTR region of F3 reduces expression of SM1 and addition of a transcription enhancer element in F2 can increase expression of SM1 above what can be achieved with an optimal 5′-UTR only.

Finally, the use of a split intron design can improve the efficiency of recombination between I1 and I2 at the pre-defined genomic location as shown in the experimental section, Example 1. One potential explanation to the improved integration efficiency observed is that the 5′-part of the split intron at the pre-defined genomic location function as a critical spacer that can avoid/reduce steric interference between RNA polymerase initiation complex binding around the start transcription site and copies of the first DNA enzyme (for example PhiC31) performing its function by binding and manipulation of I1. To increase integration efficiency, said 5′-part of a split intron can be designed to have a length of at least 50 bp, at least 100 bp or at least 300 bp.

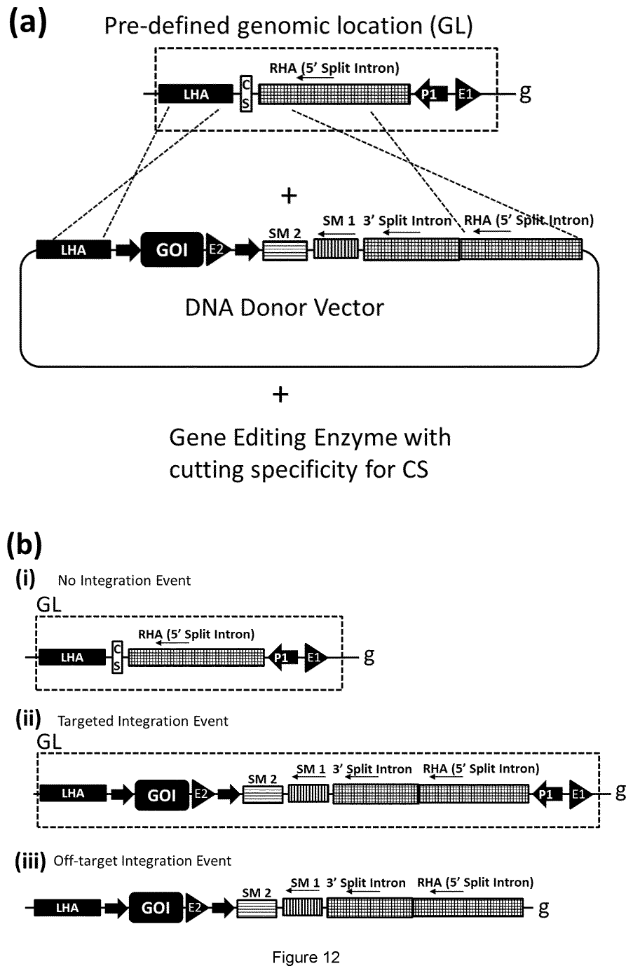

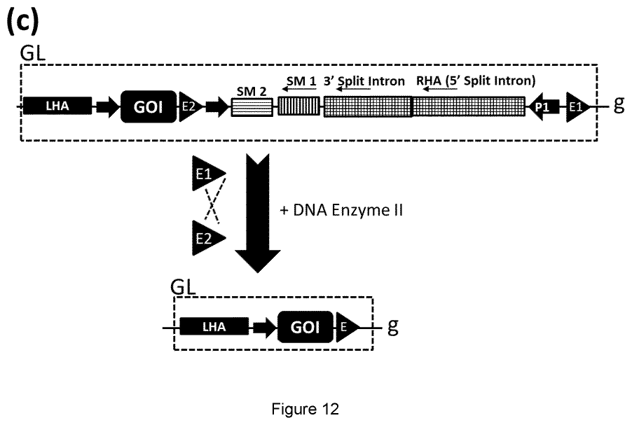

FIG. 12 illustrate a method wherein a gene editing enzyme is used to catalyze integration of the donor vector at the pre-defined genomic location (the first DNA enzyme is a gene editing enzyme). Modifications thereof according to FIG. 1, FIG. 9-11 and FIG. 15 are also encompassed by the present disclosure.

In this example the pre-defined genomic location comprise in 5′-3′ sequence order; (i) a Left Homology arm (LHA), (ii) a recognition site/Cut Site (CS) for the gene editing enzyme, (iii) a Right Homology Arm (RHA) which also function as the 5′-part of an Intron with 3′-5′ directionality (i.e. having a splice donor Site at the end closest to the promotor), (iv) a Promotor P1 with 3′-5′ directionality and (v) a recognition site E1 for a second DNA enzyme.

In this example, the donor vector comprise in 5′-3′ sequence order; (i) said Left Homology Arm (LHA), (ii) an Integration Cassette (IC), here exemplified by an expression cassette for a Gene Of Interest (GOI), (iii) a recognition site E2 for said second DNA enzyme, (iv) an expression cassette for a second Selection Marker (SM2), (v) a gene for a first Selection Marker (SM1) encoded in 3′-5′ directionality, (vi) the 3′-part of an Intron with 3′-5′ directionality (i.e. having a splice branch site and a splice acceptor site at the end closest to SM1) and (vii) said Right Homology Arm (RHA) which also function as the 5′-part of an Intron with 3′-5′ directionality.

Introduction of the donor vector and a gene editing enzyme with cut specificity for CS into a population of eukaryotic cells results in; (a) a double strand break at CS in the pre-defined genomic location for a fraction of the eukaryotic cells, (b) Integration of the donor vector region flanked by the LHA and RHA by Homology Directed DNA Repair for a fraction of eukaryotic cells having a double strand break at CS, (c) off-target genomic integration (outside the pre-defined genomic region) of the donor vector for a fraction of LP cells.

Integration at the pre-defined genomic location results in the formation of an active expression cassette for SM1 (See FIG. 12b, panel (ii)). As the integration event further creates a fully functional Intron between the Promotor P1 and SM1, mature mRNA for SM1 does not comprise the RHA. Further, following integration at the LP both SM1 and SM2 are flanked by two recognition sites for said second DNA Enzyme.

Integration by an off-target event (See FIG. 12b, panel (iii)) does not typically lead to the activation of SM1 but does integrate an active SM2 that is not flanked by two recognition sites for said second DNA Enzyme.

Eukaryotic cells having undergone integration at the pre-defined genomic location differs from cells with no integration event (See FIG. 12b, panel (i)) and cells having undergone only an off-target integration event through the activity of SM1. Hence, activity of SM1 can be used to select for cells having undergone integration at the pre-defined genomic location.

To remove cells having undergone an off-target integration event in addition to integration at the pre-defined genomic location, recombinase activity (second DNA Enzyme) capable of recombining E1 and E2 is introduced within the cells selected for SM1 activity. For integration at the LP, this results in the excision of both SM1 and SM2 and hence their corresponding activity. For off-target integration events this reaction cannot occur and SM2 activity remains. As a result, the cells having undergone only the desired targeted integration event at the pre-defined genomic location can be selected from cells having undergone a multiple integration event through absence of SM2 activity.

The pre-defined genomic location for the finally selected cells (See FIG. 12c) does not contain the expression cassette for SM2 nor the activated expression cassette for SM1 or any residual sequence from the donor vector except a sequence created through the recombination of E1 and E2 (E).

The gene editing enzyme may be selected from the groups of (i) Zinc Finger Nucleases (ZFNs); Homing Endo Nucleases such as Meganucleases; (iii) TALENs or (iv) DNA or RNA guided nucleases, such as CRISPR/Cas9, but it is not limited thereto.

Said second DNA Enzyme has recombinase activity and may be selected from the groups of (i) Serine recombinases or (ii) Tyrosine recombinases.

E1 and E2 may be identical in sequence as exemplified by; (i) E1=E2=loxP or its mutated variants for use with the Cre recombinase, (ii) E1=E2=rox or its mutated variants for use with the Dre recombinase or (iii) E1=E2=FRT or its mutated variants for use with the FLP recombinase.

E1 and E2 can have different sequences as exemplified by; (i) E1=attP and E2=attB for use with the PhiC31 recombinase or (ii) E1=Bxb1 attP and E2=Bxb1 attB.

Accordingly, there is provided herein a method, wherein said first DNA enzyme is a gene editing enzyme, such as a gene editing nuclease. Thereby there is provided a method, wherein: (a) I1 comprises a cut site for said gene editing nuclease and two sequence regions LHA1 and RHA1; and (b) I2 comprises two sequence regions LHA2 and RHA2 homologous to LHA1 and LHA2; and (c) I1 and I2 are capable of recombination in the presence of said first DNA enzyme.

As previously mentioned, there is provided a method wherein said gene editing enzyme is selected from the group consisting of (i) zink finger nucleases (ZFNs); (ii) homing endo nucleases, such as meganucleases; (iii) TALENS and (iv) DNA or RNA guided nucleases, such as CRISPR/Cas 9, but the present disclosure is not limited thereto.

The nucleic acid sequences E1 and E2 may be identical recombinase recognition sites, such as loxP, rox or FRT or variants thereof, respectively, provided that E1 and E2 are different from I1 and I2.

Said second DNA enzyme may be selected from the group consisting of a Cre recombinase, a Dre recombinase and a FLP recombinase, provided that the first DNA enzyme is not a Cre recombinase, a Dre recombinase or a FLP recombinase.

In a method provided herein, the promotor nucleic acid sequence P1 and/or P2 when integrated at said pre-defined genomic location may functionally be fused to the 5′-part of a split intron. This is illustrated in FIG. 15 previously discussed herein. The introduction of a split intron between the promoter P1 (or P2) and I1 (or a variant thereof) in the pre-defined genomic location provides a “spacer” minimizing steric hindrance which may occur due to blockage from the polymerase to the promoter. The presence of this spacer provides for an improved expression of the first selection marker (SM1) as shown in the experimental section, Example 1.

Accordingly, there is provided herein a method wherein the pre-defined genomic location further comprises the 5′-part of an Intron with 3′-5′ directionality and a functional sequence region F1 with 3′-5′ directionality between said first recognition site for said first recombinase enzyme and said promotor P1 with 3′-5′ directionality and wherein the donor vector further comprise a sequence region located between said first selection marker SM1 with 3′-5′ directionality and said second recognition site for said first recombinase enzyme. Said sequence region comprise in 5′-3′ sequence order; (a) a functional sequence region F3 with 3′-5′ directionality and (b) the 3′-part of an Intron with 3′-5′ directionality further comprising a functional sequence region F2 downstream of the splice acceptor site.

There is also provided a method herein wherein said excised nucleic acid sequence, as disclosed previously herein, comprises;

(a) a nucleic acid sequence encoding a first selection marker;

(b) a promotor nucleic acid sequence P1 or P2; and/or

(c) an expression cassette encoding a second selection marker.

The above-mentioned design of the excised nucleic acid sequence provides for the selection of cells not having randomly integrated a donor vector in other locations than the pre-defined genomic location based on the expression of a second selection marker (SM2). This means that using such a design, the expression of SM2 would be positive following action of said second DNA enzyme only for a cell having integrated the Donor vector outside the pre-defined genomic location. Accordingly, the second round of selection can use a negative selection step based on the expression of SM2 to remove cells having integrated a donor vector outside the pre-defined genomic location. The removal of the expression cassette encoding the second selection marker is also an improvement to the method as that will save energy for the cell which may instead be used for producing the protein of interest.

A first selection marker may be selected from the groups of (i) fluorescent proteins and (ii) heterologous cell surface markers, in addition to what has been mentioned elsewhere herein. The use of a fluorescent protein or a cell surface marker as a selection marker provides particular advantages as the selection can be performed using fast and direct isolation methods (based on i.e. FACS or MACS) as soon as the concentration of the first selection marker has increased above a certain limit (allowing detection of fluorescence above background in FACS and allowing efficient binding to magnetic beads in MACS). This is in contrast to selection markers based on metabolic enzymes or antibiotic resistance genes needing a prolonged and indirect isolation strategy based on cells with an activated selection marker slowly out-growing cells lacking active selection marker.

The first DNA enzyme may be provided in the form of a plasmid, mRNA or a purified protein, optionally wherein said first DNA enzyme may be encoded by and expressed from said donor vector. The first DNA enzyme may also be expressed from an expression cassette encoding said first DNA enzyme which is present in the pre-defined genomic location of a cell of step i) of a method disclosed herein.

As previously mentioned herein, a donor vector of step ii) may further comprise an expression cassette encoding a second DNA enzyme, the expression of said second DNA enzyme being activated when said donor vector has been integrated into a pre-defined genomic location of a cell of step i) in a method disclosed herein.

The second DNA enzyme may also be provided in the form of a plasmid, mRNA or a purified protein.

A eukaryotic cell for use in a method presented herein may be selected from the group consisting of a yeast cell, a filamentous fungus cell, a plant cell, an insect cell or a mammalian cell. A mammalian cell may be a human, monkey, rodent or a mouse cell, but is not limited thereto. A eukaryotic cell is an isolated eukaryotic cell as previously mentioned herein. An isolated cell is a cell that has been isolated or removed from its natural environment.

A eukaryotic cell for use in a method presented herein may specifically be selected based on suitability for production of recombinant proteins in a bioreactor. A suitable cell can be selected from the group of CHO or HEK cell lines.

A eukaryotic cell for use in a method presented herein may specifically be selected based on similarity to a cell type present in a mammalian species such as humans.

A eukaryotic cell for use in a method presented herein may be selected from the group of cell lines capable of growing in suspension cultures.

In another aspect, there is also provided an isolated eukaryotic cell obtainable by a method as disclosed herein. An isolated eukaryotic cell obtainable by a method disclosed herein contains one or more nucleic acid sequence(s) of interest integrated into the pre-defined genomic location. Preferably, the obtained isolated cell does not contain any donor vectors that have been integrated at other positions in the host cell genome than into the pre-defined genomic position.

In yet another aspect, there is also provided the use of an isolated eukaryotic cell obtainable by a method disclosed herein for the production of a recombinant protein.

Hence, there is also provided in another aspect, a method for producing a recombinant protein, said method comprising:

i) obtaining an isolated eukaryotic cell comprising one or more nucleic acid sequences of interest integrated at a pre-defined genomic location by performing a method as disclosed herein, wherein at least one nucleic acid sequence of interest comprises at least one expression cassette comprising a gene encoding a protein of interest;

ii) in said cell of step i), producing a protein encoded by the gene of interest; and

iii) isolating the protein of step ii).

In a further aspect, there is provided a donor vector comprising:

a. a nucleic acid sequence comprising a recognition site for a first DNA enzyme;

b. a nucleic acid sequence of interest;

c. a nucleic acid sequence comprising a recognition site for a second DNA enzyme;

In yet a further aspect, there is provided an isolated eukaryotic cell comprising a pre-defined genomic location, which pre-defined location comprises:

a. a nucleic acid sequence I1 comprising a recognition site for a first DNA enzyme;

b. a nucleic acid sequence E1 comprising a recognition site for a second DNA enzyme; and

c. a promotor nucleic acid sequence P1;

d. a nucleic acid sequence encoding a first selection marker;

e. optionally an expression cassette encoding a second selection marker;

In yet a further aspect, there is provided a recombinant expression system for targeted integration of a nucleic acid sequence of interest into a host cell, said expression system comprising:

a donor vector comprising:

a. a nucleic acid sequence comprising a recognition site for a first DNA enzyme;

b. a nucleic acid sequence of interest;

c. a nucleic acid sequence comprising a recognition site for a second DNA enzyme; and

an isolated eukaryotic cell comprising a pre-defined genomic location, which pre-defined location comprises:

a. a nucleic acid sequence II comprising a recognition site for a first DNA enzyme;

b. a nucleic acid sequence E1 comprising a recognition site for a second DNA enzyme; and

c. a promotor nucleic acid sequence P1;

d. a nucleic acid sequence encoding a first selection marker;

e. optionally an expression cassette encoding a second selection marker;

The present disclosure will now be illustrated by the following experimental section without being limited to the examples provided therein. The experimental section merely illustrates different ways of performing the invention.

### EXPERIMENTAL SECTION

Abbreviations

LP=Landing Pad

LP1P1=Landing Pad 1 comprising attP1

LP2P2=Landing Pad 2 comprising attP2 and a split intron

CHO=Chinese Hamster Ovary

FC-eGFP=Enhanced Green Fluorescent Protein fused to an FC from IgG1

TagBFP2=Blue Fluorescent Protein variant

TagRFP-T=Red Fluorescent Protein Variant

G418=Also known as geneticin, a broad-spectrum antibiotic that will select mammalian cells expressing the neomycin resistance gene (NeoR).

### SEQUENCE LISTING

The below sequences are used in the experimental section, but the present disclosure is not limited to these sequences. Accordingly, variants thereof are also envisaged wherein the function of said sequence variants remain essentially the same as the original sequence.

attP1
(SEQ ID NO: 1)
GTGCCCCAACTGGGGTAACCTTTGAGTTCTCTCAGTTGGGGGCGTAG
attB1
(SEQ ID NO: 2)
CTCGAAGCCGCGGTGCGGGTGCCAGGGCGTGCCCTTGGGCTCCCCGG
GCGCGTACTCCACCTCACCCATC
attP2
(SEQ ID NO: 3)
GTGCCCCAACTGGGGTAACCTAAGAGTTCTCTCAGTTGGGGGCGTAG
attB2
(SEQ ID NO: 4)
CTCGAAGCCGCGGTGCGGGTGCCAGGGCGTGCCCAAGGGCTCCCCGG
GCGCGTACTCCACCTCACCCATC
PhiC31 gene
(SEQ ID NO: 5)
ATGACCATGATTACCCCATCTGCCCAGCTGACCCTGACAAAGGGCAA
TAAGAGCTGGTCTAGCCTGGTGACAGCTGCTTCTGTGCTGGAGTTTG
CCACCATGATCCAAGGGGTCGCTGGGGAAGTGACTTATGCCGGGGCG
TACGACCGTCAGTCTCGGGAGCGCGAGAACTCTAGCGCGGCGTCTCC
GGCCACTCAGCGTAGCGCTAACGAGGCCAAAGCCGCCGCTCTCCAGC
GCGAGATCGAGCGCGCCGGGGGCCGGTTTCGTTTCGTCGGTCACTTC
AGCGAGGCCCCCGGCACATCTGCCTTCGGTACAGCCGAGCGCCCTGA
GTTCGAACGCATTCTGAACGAATGCCGCGCCGGTCGGCTGAACATGA
TTATCGTGTATGACGTGTCTCGCTTCTCTCGCCTGAAGGTTATGGAC
GCCATCCCTATCGTGTCAGAATTACTGGCCCTGGGCGTGACAATCGT
CTCTACGCAGGAAGGCGTGTTCAGACAAGGGAACGTTATGGACCTGA
TCCACCTGATCATGCGGCTGGACGCCTCTCACAAAGAAAGCTCTCTG
AAGTCTGCCAAGATCCTGGACACAAAGAACCTCCAGCGCGAACTTGG
CGGTTACGTGGGCGGGAAGGCCCCCTACGGCTTCGAGCTTGTCAGCG
AGACAAAGGAGATTACACGCAACGGACGTATGGTCAATGTGGTTATC
AACAAGCTCGCCCACTCTACCACGCCTCTCACCGGACCTTTCGAGTT
CGAGCCAGACGTAATTCGGTGGTGGTGGCGTGAGATCAAGACACACA
AACACCTCCCTTTCAAGCCTGGCAGTCAAGCCGCCATCCACCCTGGC
TCTATTACCGGACTCTGTAAGCGCATGGACGCGGACGCCGTGCCTAC
CAGAGGCGAGACAATCGGGAAGAAGACCGCGTCGTCTGCCTGGGACC
CTGCGACCGTCATGCGTATTCTCAGAGACCCTCGTATCGCCGGGTTC
GCTGCGGAGGTGATTTACAAGAAGAAGCCAGACGGCACACCTACCAC
AAAGATCGAGGGATACCGCATCCAGCGCGACCCTATTACTCTGCGGC
CTGTGGAGCTTGATTGCGGTCCTATTATCGAGCCTGCGGAGTGGTAT
GAGCTTCAGGCCTGGTTGGACGGACGTGGTCGCGGCAAGGGTCTCTC
TCGGGGTCAAGCCATCCTGTCTGCTATGGACAAGCTGTACTGCGAGT
GTGGCGCCGTTATGACGAGCAAGCGCGGGGAAGAATCTATCAAGGAC
AGTTACCGCTGCCGTCGCAGAAAGGTGGTGGACCCTTCTGCGCCCGG
TCAGCACGAAGGCACTTGCAACGICICIAIGGCCGCGCTGGACAAGT
TCGTCGCCGAACGCATTTTCAACAAGATCCGTCACGCCGAAGGCGAC
GAAGAGACACTTGCCCTCCTGTGGGAAGCCGCCCGTCGCTTCGGCAA
GCTCACGGAGGCCCCCGAGAAGTCTGGCGAAAGAGCCAACCTCGTCG
CCGAGCGCGCCGACGCCCTGAACGCCCTCGAAGAGCTGTACGAAGAC
CGCGCTGCGGGCGCCTACGACGGTCCTGTCGGACGAAAGCACTTCAG
AAAGCAACAGGCGGCCCTGACTCTGCGCCAGCAAGGTGCCGAAGAGA
GACTCGCCGAACTCGAAGCCGCCGAAGCCCCAAAGCTCCCTCTCGAC
CAATGGTTCCCAGAAGACGCCGACGCGGACCCTACCGGCCCCAAGTC
TTGGTGGGGTCGCGCCTCGGTAGACGACAAGCGCGTGTTCGTGGGTC
TGTTCGTAGACAAGATTGTCGTTACAAAGTCTACGACAGGCCGTGGG
CAGGGGACACCTATCGAGAAGCGCGCGTCTATTACTTGGGCCAAGCC
TCCTACCGACGACGACGAAGACGACGCCCAGGACGGCACAGAAGACG
TAGCTGCTTGATAA
IoxP
(SEQ ID NO: 6)
ATAACTTCGTATAGGATACTTTATACGAAGTTAT
Cre gene
(SEQ ID NO: 7)
ATGTCAAACCTTCTCACCGTCCACCAAAACCTCCCCGCACTCCCCGT
TGACGCCACCTCCGACGAGGTCAGAAAAAACCTCATGGACATGTTCC
GGGACCGCCAGGCCTTTTCCGAACACACTTGGAAAATGCTTCTCAGC
GTTTGCCGTAGTTGGGCCGCTTGGTGTAAACTCAACAACCGCAAGTG
GTTCCCCGCCGAACCCGAGGACGTCCGCGATTACCTTCTGTATTTGC
AAGCGCGAGGACTGGCCGTGAAAACCATCCAGCAACATCTGGGTCAG
CTTAACATGTTGCACCGGAGGAGCGGCCTGCCACGGCCTAGCGACTC
CAACGCGGTGTCCCTCGTGATGAGGAGAATCCGCAAGGAGAATGTGG
ACGCCGGAGAAAGAGCAAAGCAGGCCCTGGCCTTCGAGAGGACTGAC
TTCGACCAAGTCCGGTCGCTGATGGAGAACTCGGACCGATGTCAGGA
CATCAGGAACCTCGCATTCTCGGCATTGCCTACAACACCCTGCTGAG
AATTGCAGAGATCGCCCGCATCCGCGTCAAGGACATTTCGAGAACCG
ACGGAGGGCGGATGCTGATTCACATCGGCAGGACTAAGACCCTCGTG
TCAACCGCCGGAGTGGAAAAGGCCCTCAGCCTGGGAGTGACAAAGCT
CGTGGAGCGCTGGATCTCCGTGTCGGGGGTGGCCGACGATCCGAACA
ATTACCTGTTCTGCCGGGTCCGCAAAAATGGGGTGGCCGCCCCGTCT
GCTACAAGCCAGTTGTCCACTCGCGCCCTGGAAGGAATCTTCGAGGC
CACGCACCGCCTGATCTATGGGGCAAAGGACGATTCCGGCCAGAGGT
ATCTCGCGTGGTCCGGTCACTCCGCGCGCGTGGGCGCGGCCCGGGAC
ATGGCCCGGGCTGGAGTGTCCATCCCTGAAATCATGCAGGCCGGTGG
ATGGACCAACGTGAACATCGTGATGAACTACATTCGGAACCTGGACA
GCGAAACTGGTGCTATGGTCCGCCTGCTGGAGGACGGAGATTGA
FC-eGFP gene
(SEQ ID NO: 8)
GACAAAACTCACACATGCCCACCGTGCCCAGCACCTGAACTCCTGGG
GGGACCGTCAGTCTTCCTCTTCCCCCCAAAACCCAAGGACACCCTCA
TGATCTCCCGGACCCCTGAGGTCACATGCGTGGTGGTGGACGTGAGC
CACGAAGACCCTGAGGTCAAGTTCAACTGGTACGTGGACGGCGTGGA
GGTGCATAATGCCAAGACAAAGCCACGGGAGGAGCAGTACAACAGCA
CGTACCGTGTGGTCAGCGTCCTCACCGTCCTGCACCAGGACTGGCTG
AATGGCAAGGAGTACAAGTGCAAGGTCTCCAACAAAGCCCTCCCAGC
CCCCATCGAGAAAACCATCTCCAAAGCCAAAGGGCAGCCCCGAGAAC
CACAGGTCTACACCCTGCCCCCATCCCGGGAGGAGATGACCAAGAAC
CAGGTCAGCCTGACCTGCCTGGTCAAAGGCTTCTATCCCAGCGACAT
CGCCGTGGAGTGGGAGAGCAATGGGCAGCCGGAGAACAACTACAAGA
CCACGCCTCCCGTGCTGGACTCCGACGGCTCCTTCTTCCTCTACAGC
AAGCTCACCGTGGACAAGAGCAGGTGGCAGCAGGGGAACGTCTTCTC
ATGCTCCGTGATGCACGAGGCTCTGCACAACCACTACACGCAGAAGA
GCCTCTCCCTGTCTCCGGGTAAAGGTTCCTCCAGTTCCGGCAGCTCC
AGTTCCGGTATGAGTAAAGGAGAGGAACTCTTCACCGGAGTCGTCCC
GATACTCGTCGAGCTAGACGGAGACGTCAACGGCCACAAATTCTCCG
TCTCCGGCGAGGGGGAGGGGGACGCCACCTACGGAAAACTCACCCTT
AAGTTTATTTGCACTACCGGAAAACTCCCCGTCCCTTGGCCAACCCT
AGTCACCACGCTGACATACGGAGTCCAATGTTTCTCGCGGTATCCCG
ACCACATGAAGCAGCATGACTTTTTCAAATCCGCGATGCCTGAGGGC
TACGTGCAGGAACGCACCATCTTCTTCAAGGACGACGGGAATTACAA
GACTAGAGCCGAGGTCAAGTTTGAAGGAGACACCCTCGTGAATCGCA
TCGAGCTTAAGGGCATTGACTTCAAGGAGGACGGCAACATCCTGGGT
CACAAGCTGGAGTACAACTACAACTCGCATAACGTCTACATCATGGC
CGACAAGCAAAAGAACGGTATCAAGGTCAACTTCAAGATTAGGCACA
ACATTGAGGATGGGTCCGTCCAACTGGCCGACCACTACCAGCAGAAC
ACCCCCATCGGCGACGGACCTGTGCTCCTGCCTGATAACCACTATCT
CAGCACTCAGAGCGCACTGTCCAAGGACCCTAACGAAAAACGGGACC
ACATGGTCTTGCTGGAGTTCGTGACAGCCGCTGGTATTACCCTGGGC
ATGGATGAACTGTATAAG
FC-TagBFP2 gene
(SEQ ID NO: 9)
GACAAAACTCACACATGCCCACCGTGCCCAGCACCTGAACTCCTGGG
GGGACCGTCAGTCTTCCTCTTCCCCCCAAAACCCAAGGACACCCTCA
TGATCTCCCGGACCCCTGAGGTCACATGCGTGGTGGTGGACGTGAGC
CACGAAGACCCTGAGGTCAAGTTCAACTGGTACGTGGACGGCGTGGA
GGTGCATAATGCCAAGACAAAGCCACGGGAGGAGCAGTACAACAGCA
CGTACCGTGTGGTCAGCGTCCTCACCGTCCTGCACCAGGACTGGCTG
AATGGCAAGGAGTACAAGTGCAAGGTCTCCAACAAAGCCCTCCCAGC
CCCCATCGAGAAAACCATCTCCAAAGCCAAAGGGCAGCCCCGAGAAC
CACAGGTCTACACCCTGCCCCCATCCCGGGAGGAGATGACCAAGAAC
CAGGTCAGCCTGACCTGCCTGGTCAAAGGCTTCTATCCCAGCGACAT
CGCCGTGGAGTGGGAGAGCAATGGGCAGCCGGAGAACAACTACAAGA
CCACGCCTCCCGTGCTGGACTCCGACGGCTCCTTCTTCCTCTACAGC
AAGCTCACCGTGGACAAGAGCAGGTGGCAGCAGGGGAACGTCTTCTC
ATGCTCCGTGATGCACGAGGCTCTGCACAACCACTACACGCAGAAGA
GCCTCTCCCTGTCTCCGGGTAAAGGTTCCTCCAGTTCCGGCAGCTCC
AGTTCCGGTATGGTGTCGAAGGGAGAGGAGCTGATTAAGGAGAACAT
GCACATGAAGCTGTATATGGAAGGGACGGTGGACAACCACCACTTCA
AGTGCACCAGCGAAGGAGAAGGAAAGCCTTACGAAGGCACTCAAACT
ATGCGGATCAAAGTGGTGGAAGGCGGTCCTCTTCCGTTCGCCTTCGA
CATCTTGGCCACCTCCTTCCTCTACGGCTCCAAGACCTTTATCAACC
ACACCCAGGGAATCCCGGACTTCTTTAAGCAGAGCTTCCCTGAGGGC
TTCACCTGGGAAAGAGTGACAACCTACGAGGACGGTGGCGTCCTGAC
CGCGACCCAGGACACCTCCCTGCAAGACGGCTGCCTGATCTACAACG
TCAAGATTCGCGGCGTGAACTTCACCTCCAATGGTCCAGTGATGCAG
AAGAAAACTCTGGGATGGGAGGCCTTCACTGAAACTCTGTACCCCGC
CGATGGAGGACTGGAGGGGAGGAACGATATGGCTTTGAAGCTCGTGG
GGGGATCGCACCTGATTGCGAATGCCAAGACCACCTACAGATCCAAG
AAACCCGCCAAGAACCTCAAGATGCCCGGAGTCTACTACGTGGACTA
TAGACTGGAACGGATCAAGGAAGCCAACAACGAGACTTACGTGGAAC
AGCACGAGGTCGCTGTGGCACGCTACTGTGATCTGCCGTCAAAGCTC
GGGCATAAGCTCAACTGATAA
TagRFP-T gene
(SEQ ID NO: 10)
ATGGTGTCAAAGGGAGAGGAACTGATTAAGGAGAATATGCACATGAA
ACTCTACATGGAGGGGACCGTGAACAACCACCACTTCAAGTGCACCT
CCGAGGGCGAAGGGAAGCCGTACGAGGGAACTCAGACCATGCGGATT
AAGGTCGTCGAAGGGGGTCCTCTGCCATTCGCCTTCGACATCCTCGC
CACATCCTTTATGTACGGATCGCGGACCTTCATCAACCACACTCAGG
GTATCCCCGACTTCTTCAAGCAATCGTTCCCGGAAGGCTTTACTTGG
GAGCGCGTGACCACCTACGAGGATGGAGGGGTGCTGACGGCCACTCA
GGACACCAGCCTGCAAGACGGCTGTCTTATCTACAACGTGAAGATTC
GCGGCGTGAACTTCCCTAGCAACGGTCCGGTCATGCAGAAAAAGACC
CTGGGTTGGGAGGCTAACACCGAAATGCTCTATCCTGCGGACGGAGG
ATTGGAAGGCCGGACTGACATGGCCCTGAAACTTGTGGGCGGCGGAC
ATCTGATCTGCAATTTCAAGACCACTTACCGCTCCAAGAAGCCCGCC
AAGAACCTGAAGATGCCTGGAGTGTACTACGTGGACCACAGACTCGA
AAGGATCAAGGAGGCGGATAAGGAAACCTACGTGGAACAGCATGAAG
TGGCAGTGGCCAGATACTGCGATCTGCCGTCCAAGCTCGGCCACAAG
CTGAACGGAATGGACGAGCTGTATAAGTGATAA
eGFP gene
(SEQ ID NO: 11)
ATGAGTAAAGGAGAGGAACTCTTCACCGGAGTCGTCCCGATACTCGT
CGAGCTAGACGGAGACGTCAACGGCCACAAATTCTCCGTCTCCGGCG
AGGGGGAGGGGGACGCCACCTACGGAAAACTCACCCTTAAGTTTATT
TGCACTACCGGAAAACTCCCCGTCCCTTGGCCAACCCTAGTCACCAC
GCTGACATACGGAGTCCAATGTTTCTCGCGGTATCCCGACCACATGA
AGCAGCATGACTTTTTCAAATCCGCGATGCCTGAGGGCTACGTGCAG
GAACGCACCATCTTCTTCAAGGACGACGGGAATTACAAGACTAGAGC
CGAGGTCAAGTTTGAAGGAGACACCCTCGTGAATCGCATCGAGCTTA
AGGGCATTGACTTCAAGGAGGACGGCAACATCCTGGGTCACAAGCTG
GAGTACAACTACAACTCGCATAACGTCTACATCATGGCCGACAAGCA
AAAGAACGGTATCAAGGTCAACTTCAAGATTAGGCACAACATTGAGG
ATGGGTCCGTCCAACTGGCCGACCACTACCAGCAGAACACCCCCATC
GGCGACGGACCTGTGCTCCTGCCTGATAACCACTATCTCAGCACTCA
GAGCGCACTGTCCAAGGACCCTAACGAAAAACGGGACCACATGGTCT
TGCTGGAGTTCGTGACAGCCGCTGGTATTACCCTGGGCATGGATGAA
CTGTATAAG
TagBFP2 gene
(SEQ ID NO: 12)
ATGGTGTCGAAGGGAGAGGAGCTGATTAAGGAGAACATGCACATGAA
GCTGTATATGGAAGGGACGGTGGACAACCACCACTTCAAGTGCACCA
GCGAAGGAGAAGGAAAGCCTTACGAAGGCACTCAAACTATGCGGATC
AAAGTGGTGGAAGGCGGTCCTCTTCCGTTCGCCTTCGACATCTTGGC
CACCTCCTTCCTCTACGGCTCCAAGACCTTTATCAACCACACCCAGG
GAATCCCGGACTTCTTTAAGCAGAGCTTCCCTGAGGGCTTCACCTGG
GAAAGAGTGACAACCTACGAGGACGGTGGCGTCCTGACCGCGACCCA
GGACACCTCCCTGCAAGACGGCTGCCTGATCTACAACGTCAAGATTC
GCGGCGTGAACTTCACCTCCAATGGTCCAGTGATGCAGAAGAAAACT
CTGGGATGGGAGGCCTTCACTGAAACTCTGTACCCCGCCGATGGAGG
ACTGGAGGGGAGGAACGATATGGCTTTGAAGCTCGTGGGGGGATCGC
ACCTGATTGCGAATGCCAAGACCACCTACAGATCCAAGAAACCCGCC
AAGAACCTCAAGATGCCCGGAGTCTACTACGTGGACTATAGACTGGA
ACGGATCAAGGAAGCCAACAACGAGACTTACGTGGAACAGCACGAGG
TCGCTGTGGCACGCTACTGTGATCTGCCGTCAAAGCTCGGGCATAAG
CTCAACTGATAA
NeoR gene
(SEQ ID NO: 13)
ATGATTGAACAAGATGGATTGCACGCAGGTTCTCCGGCCGCTTGGGT
GGAGAGGCTATTCGGCTATGACTGGGCACAACAGACAATCGGCTGCT
CTGATGCCGCCGTGTTCCGGCTGTCAGCGCAGGGGCGCCCGGTTCTT
TTTGTCAAGACCGACCTGTCCGGTGCCCTGAATGAACTGCAGGACGA
GGCAGCGCGGCTATCGTGGCTGGCCACGACGGGCGTTCCTTGCGCAG
CTGTGCTCGACGTTGTCACTGAAGCGGGAAGGGACTGGCTGCTATTG
GGCGAAGTGCCGGGGCAGGATCTCCTGTCATCTCACCTTGCTCCTGC
CGAGAAAGTATCCATCATGGCTGATGCAATGCGGCGGCTGCATACGC
TTGATCCGGCTACCTGCCCATTCGACCACCAAGCGAAACATCGCATC
GAGCGAGCACGTACTCGGATGGAAGCCGGTCTTGTCGATCAGGATGA
TCTGGACGAAGAGCATCAGGGGCTCGCGCCAGCCGAACTGTTCGCCA
GGCTCAAGGCGCGCATGCCCGACGGCGAGGATCTCGTCGTGACCCAT
GGCGATGCCTGCTTGCCGAATATCATGGTGGAAAATGGCCGCTTTTC
TGGATTCATCGACTGTGGCCGGCTGGGTGTGGCGGACCGCTATCAGG
ACATAGCGTTGGCTACCCGTGATATTGCTGAAGAGCTTGGCGGCGAA
TGGGCTGACCGCTTCCTCGTGCTTTACGGTATCGCCGCTCCCGATTC
GCAGCGCATCGCCTTCTATCGCCTTCTTGACGAGTTCTTC

### Example 1: Efficiency of phiC31 Recombinase-Mediated Integration and Selection Marker Activation

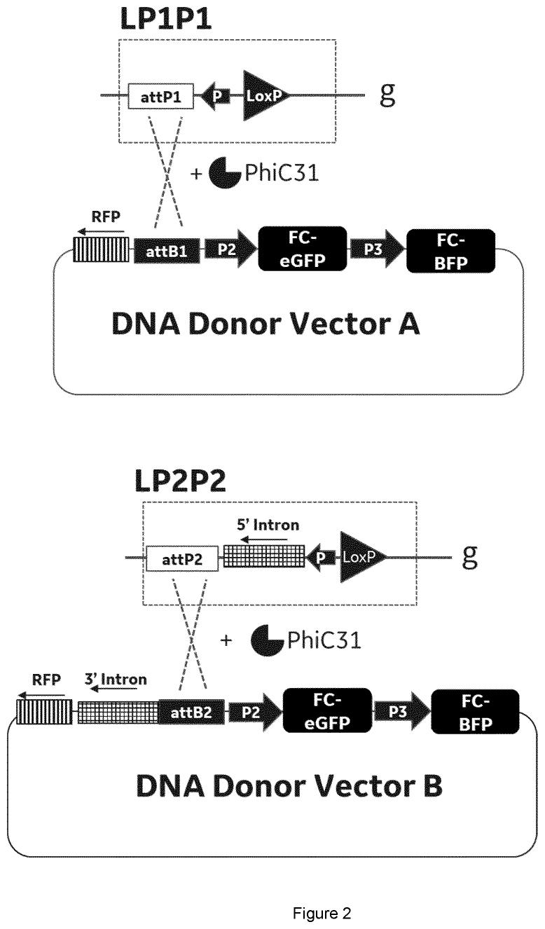

To investigate the efficiency of integration, HyClone CHO LP cells and non-LP HyClone CHO control cells were transfected with a combination of PhiC31 recombinase expression plasmid and either of Donor vector A or B (FIG. 2). The Donor vectors contain expression cassettes for FC-eGFP and FC-TagBFP2 and a promoter-less TagRFP-T gene positioned so that it activates upon integration at the LP in LP cells.

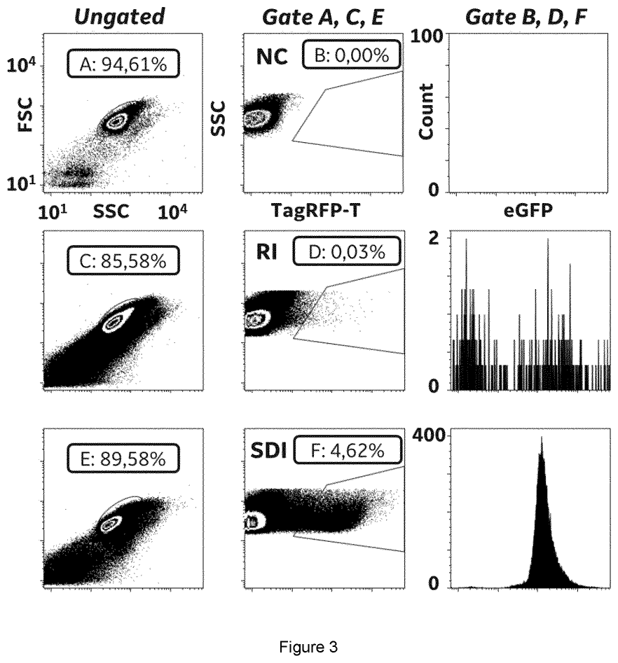

Two HyClone-CHO LP variants and matching Donor vectors were investigated, see FIG. 2. In HyClone-CHO LP1P1, the promotor in the LP is positioned directly downstream of attP1. In Donor vector A, the TagRFP-T gene is positioned directly upstream of attB1. In HyClone-CHO LP2P2, the 5′-part of a split intron is positioned between attP2 and the downstream promotor at the LP. In Donor vector B, the 3′-part of a split intron is positioned between attB2 and the upstream TagRFP-T gene. Efficiency of integration was evaluated by flow cytometry 7 days post transfection by measuring the percentage of cells displaying RFP signal above background (defined in comparison to non-transfected control, see FIG. 3).

In FIG. 3 an example of the flow cytometry data generated is illustrated for HyClone CHO LP2P2 in comparison to controls. The full set of results are summarized in Table 1. For LP1P1 only a non-transfected control was used whereas for LP2P2 both a random Integration control (RI Control, Donor vector only) and a pseudo-att integration control (Donor vector+PhiC31 in a CHO cell line lacking the LP) were performed. According to the data both LP variants are functional but the LP2P2 variant utilizing a split intron design gives superior integration efficiency.

TABLE 1
Summary of results obtained from Control experiments
and SDI integration efficiency evaluations for
both HyClone CHO LP1P1 and HyClone CHO LP2P2.

| | | | TagRFP-T |
|---|---|---|---|
| Experiment | Cell Line | Plasmids | positive cells |
| Non-transfected | HyClone CHO | — | 0% |
| Control | | | |
| LP1P1 SDI | HyClone CHO | PhiC31 + Donor A | 0.38% |
| | LP1P1 | | |
| RI Control | HyClone CHO | Donor B | 0.03% |
| Pseudo-att Control | HyClone CHO | PhiC31 + Donor B | 0.19% |
| LP2P2 SDI | HyClone CHO | PhiC31 + Donor B | 4.62% |
| | LP2P2 | | |

### Example 2: Efficiency of Cre Recombinase-Mediated Excision of Vector Backbone at the Integration Site

HyClone CHO LP1P1 cells were transfected using a PhiC31 expression plasmid and a Donor Vector containing expression cassettes for FC-eGFP and FC-TagBFP2 and a promoter-less TagRFP-T gene positioned so that it activates upon integration at the LP in LP cells (FIG. 4). Cells having integrated the Donor Vector at the Landing Pad (LP) were enriched by several FACS sorting steps gating for Tag-RFP-T signal above background and a balanced expression of both FC-eGFP and FC-TagBFP2. The resulting sorted and expanded pool of cells was then transfected a second time using either (a) a Cre recombinase expression plasmid, (b) a synthetic mRNA encoding Cre or (c) a mock transfection solution lacking any Cre recombinase encoding nucleic acid molecule. Seven days post the second transfection all cell populations were analyzed by flow cytometry to evaluate the efficiency of excision of the region flanked by two loxP sites.

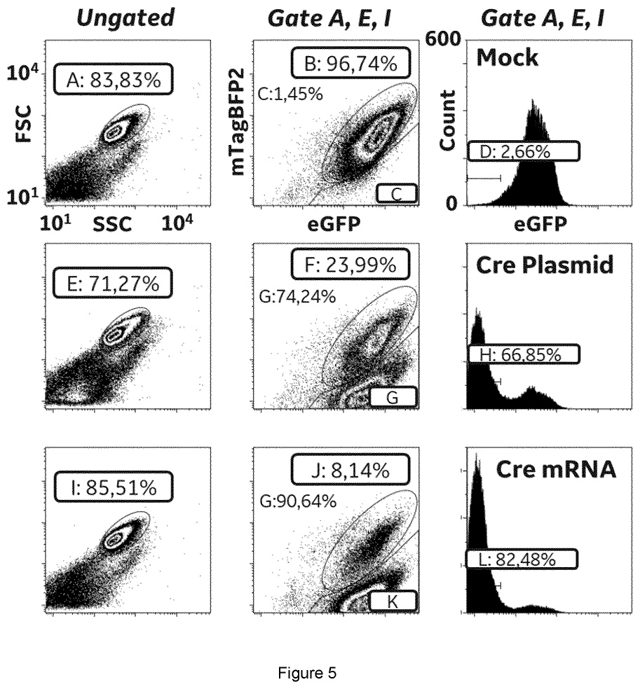

Plots from the flow cytometry analysis following the Cre recombinase transfection can be seen in FIG. 5. The data shows an increase of cells that do not express FC-TagBFP2 for the two Cre recombinase treated pools as compared with the mock control. This in turn clearly indicates correct integration of the donor vector at the LP so that FC-TagBFP2 are flanked by two loxP sites which the Cre recombinase enzyme can act on. According to the data, the excision reaction catalyzed by Cre recombinase is highly effective with a yield up to at least 80%.

### Example 3: Repeated Integration at the Same Genomic Location by the Using of Orthogonal attP/attB Pairs

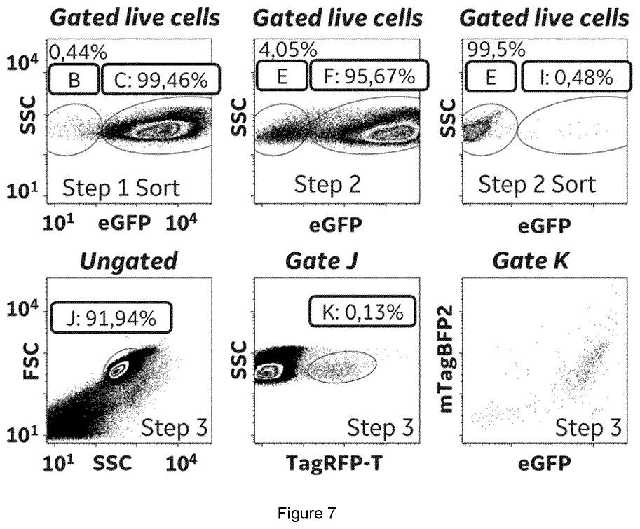

HyClone CHO LP1P1 cells were transfected using a PhiC31 expression plasmid and a Donor Vector containing an attB1 sequence followed by an attP2 sequence, the 5′-part of a split intron, a promotor, a loxP sequence and an expression cassette for eGFP (FIG. 6, Donor Vector A). At day 7 post transfection, eGFP positive cells were sorted by FACS followed by expansion of cells. A second more stringent sort of eGFP positive cells were then performed. Cells expanded after the second eGFP positive sort (FIG. 7, Step 1 Sort) were transfected using a synthetic mRNA encoding Cre recombinase. Seven days post the Cre recombinase transfection, eGFP negative cells were sorted using FACS. Following expansion, the eGFP negative cell pool was analyzed by flow cytometry (FIG. 7, Step 2 Sort). Data from the sorting and analysis steps are shown in FIG. 7, upper panel. During these steps the Landing Pad in the CHO genome is assumed to have been altered as indicated by steps (1) and (2) in FIG. 6.

To verify functionality of the altered Landing Pad, the eGFP negative pool obtained after the final sort (FIG. 7, Step 2 Sort) were transfected using DNA Donor Vector B (FIG. 6, step 3) and analyzed by flow cytometry 7 days post transfection (FIG. 7, lower panel). Data indicates functionality of the new Landing pad. Finally, cells from the eGFP negative pool were cloned using single cell sorting by FACS and the Landing Pad region of their genomes amplified by PCR and sequenced. Correct alteration of the Landing Pad was confirmed for multiple clones by sequencing (full coverage of new Landing Pad region) showing that the alteration outlined in FIG. 7 has been successfully achieved.

### Example 4: Generation of an SDI Cell Pool Using Two Consecutive Selection Steps

HyClone CHO LP2P2 cells (Clones generated according to Example 3) were transfected using a PhiC31 recombinase expression plasmid and a Donor Vector constructed according to FIG. 8.

Starting at two days post transfection, cells were cultured in the presence of G418 to select for cells having integrated the Donor Vector at the Landing Pad and thereby activated the neomycin resistance gene (Neo^{R}). Following return to high viabilities (>98%) post G418 selection, the cells were sorted by FACS based on GFP/RFP double positive signal. Following expansion, the sorted cells were transfected using synthetic mRNA encoding Cre recombinase. At day 7 post Cre recombinase transfection, cells were FACS sorted by GFP-positive/RFP-negative signal (FIG. 9, gate E). The final SDI pool was analyzed by flow cytometry following expansion.

Data from FACS/Flow Cytometry can be seen in FIG. 9. The additional selection step following Cre recombinase transfection reduces the heterogeneity in the pool as indicated by the mean value and CV of the eGFP signal for TagRFP-T positive (incorrect integration) and TagRFP-T negative (correct integration) cells.

### REFERENCES

[1] Kunert, R, et. al.; Advances in recombinant antibody manufacturing; Appl Microbiol Biotechnol (2016) 100:3451-3461.
[2] Ecker, D M, et al; The therapeutic monoclonal antibody market; mAbs 7:1, 9-14; January/February 2015.
[3] Meinke, G, et al; Cre Recombinase and Other Tyrosine Recombinases; Chem. Rev. 2016, 116, 12785-12820.
[4] Merrick, C A; et al.; Serine Integrases: Advancing Synthetic Biology; ACS Synth. Biol. 2018, 7, 299-310.
[5] Xu, Z, et al.; Accuracy and efficiency define Bxb1 integrase as the best of fifteen candidate serine recombinases for the integration of DNA into the human genome; BMC Biotechnology 2013, 13:87.
[6] Lee, J S, et al.; Accelerated Homology-Directed Targeted Integration of Transgenes in Chinese Hamster Ovary Cells Via CRISPR/Cas9 and Fluorescent Enrichment; Biotechnol. Bioeng. 2016; 9999: 1-6.
[7] Invitrogen; Flp-In system for generating stable mammalian expression cell lines by Flp recombinase-mediated integration; Invitrogen Instruction Manual 2001; Invitrogen, Carlsbad Calif.
[8] Muller, D; Accelerating Time to Clinical Manufacturing Following a Targeted Gene Integration Approach; Bioprocess International Conference, Boston; Oct. 28, 2015.
[9] Haghighat-Khah, R E, et al.; Site-Specific Cassette Exchange Systems in the Aedes aegypti Mosquito and the Plutella xylostella Moth; PLOS ONE DOI:10.1371/journal.pone.0121097 Apr. 1, 2015.
[10] Yuan, Y, et al.; Improved site-specific recombinase-based method to produce selectable marker- and vector-backbone-free transgenic cells; SCIENTIFIC REPORTS|4:4240|DOI: 10.1038/srep04240.

1. A method for targeted integration of a donor vector into a pre-defined genomic location of an isolated eukaryotic cell, said method comprising:
i) Providing an isolated eukaryotic cell comprising a pre-defined genomic location, which pre-defined location comprises:
a. a nucleic acid sequence I1 comprising a recognition site for a first DNA enzyme;
b. a nucleic acid sequence E1 comprising a recognition site for a second DNA enzyme; and
c. a promotor nucleic acid sequence P1;
ii) Providing a donor vector comprising:
a. a nucleic acid sequence I2;
b. a nucleic acid sequence of interest;
c. a nucleic acid sequence E2 comprising a recognition site for said second DNA enzyme;
d. a nucleic acid sequence encoding a first selection marker;
e. optionally an expression cassette encoding a second selection marker;
iii) Contacting the donor vector with the cell in the presence of a first DNA enzyme, wherein the presence of the first DNA enzyme enables recombination between the nucleic acid sequence I2 of the donor vector and the nucleic acid sequence I1 present in the pre-defined genomic location of the cell;
iv) Selecting a cell having the donor vector integrated at the pre-defined genomic location by detecting the expression of the first selection marker in the cell, wherein the expression of the first selection marker is activated by the promotor nucleic acid sequence P1 at the pre-defined genomic location; and
v) Isolating the cell selected in the preceding step.

2. The method of claim 1, further comprising a step vi), comprising excising a nucleic acid sequence flanked by the nucleic acid sequences E1 and E2 from the pre-defined genomic location of the cell isolated in step v) in the presence of a second DNA enzyme, wherein the presence of the second DNA enzyme enables recombination between the nucleic acid sequences E1 and E2, wherein the presence of a nucleic acid sequence flanked by the nucleic acid sequences E1 and E2 in said cell is indicative of a stable integration of the donor vector into the pre-defined genomic location of the cell.

3. The method of claim 2, wherein step vi) forms part of step iii), or is performed after step iii), such as after step iv) or after step v).

4. The method of claim 3, wherein step vi) is performed before step v).

5. The method of claim 4, wherein the donor vector of step ii) further comprises e) an expression cassette encoding a second selection marker and wherein the cell isolated in step v) additionally has been selected based on its non-expression of the second selection marker, wherein expression of the second selection marker signals that a donor vector has been integrated at a different location than the predefined genomic location of a cell.

6. The method of claim 3, wherein step vi) is performed after step v), and the method further comprises a step vii), performed after step vi), comprising isolating a cell in which the nucleic acid sequence being flanked by the nucleic acid sequences E1 and E2 has been excised from the pre-defined genomic location of the cell isolated in step vi).

7. The method of claim 6, wherein the donor vector of step ii) further comprises e) an expression cassette encoding a second selection marker, and wherein the cell isolated in step vii) has been selected based on its non-expression of the second selection marker, wherein expression of the second selection marker signals that a donor vector has been integrated at a different location than the predefined genomic location of a cell.

8. The method of claim 1, wherein the nucleic acid sequence of interest of the donor vector of step ii) comprises at least one expression cassette comprising a gene encoding a protein of interest.

9. The method of claim 8, wherein the excised nucleic acid sequence lacks the at least one expression cassette containing a gene encoding a protein of interest.

10. The method of claim 1, wherein the donor vector of step ii) further comprises:
f. a nucleic acid sequence I3 comprising a recognition site for a first DNA enzyme; and
g. a promotor nucleic acid sequence P2.

11. The method of claim 10, further comprising performing a sequential targeted integration of n additional donor vectors into the pre-defined genomic location of the eukaryotic cell,
wherein n is an integer ≥1;
the method comprising:
(A) Integrating a first additional donor vector into the pre-defined genomic location of the cell, comprising:
VII. Providing the cell isolated in step v) or isolated in step vii);
IX. Providing a donor vector comprising:
A. a nucleic acid sequence I4, which in the presence of said first DNA enzyme is capable of recombining with the corresponding nucleic acid sequence I3 present in the cell provided in the preceding step;
B. a nucleic acid sequence of interest;
C. a nucleic acid sequence E2;
D. a nucleic acid sequence encoding a first selection marker; and
E. optionally an expression cassette encoding a second selection marker;
F. optionally a nucleic acid sequence I1 comprising a recognition site for a first DNA enzyme and a promotor nucleic acid sequence P1;
X. Introducing the donor vector of step II) into the cell in the presence of a first DNA enzyme, wherein the presence of the first DNA enzyme enables recombination between the nucleic acid sequence I4 of the donor vector and the nucleic acid sequence I3 present in the pre-defined genomic location of the cell;
XI. Selecting a cell having the donor vector integrated at the pre-defined genomic location by detecting the expression of the first selection marker in the cell, wherein the expression of the first selection marker is activated by the promotor nucleic acid sequence P2 at the pre-defined genomic location of the cell;
XII. Isolating the cell selected in the preceding step;
XIII. Excising a nucleic acid sequence flanked by the nucleic acid sequences E1 and E2 from the pre-defined genomic location of the cell isolated in step V in the presence of a second DNA enzyme, wherein the presence of the second DNA enzyme enables recombination between the nucleic acid sequences E1 and E2, wherein the presence of a nucleic acid sequence flanked by the nucleic acid sequences E1 and E2 is in said cell is indicative of a stable integration of the donor vector into the pre-defined genomic location of the cell;
XIV. Isolating a cell in which the nucleic acid sequence being flanked by the sequences E1 and E2 has been excised from said pre-defined genomic location of the cell isolated in step V;
(B) Provided that n is larger than the number of additional donor vectors integrated at the pre-defined genomic location of the cell isolated in the preceding step, integrating an additional donor vector into the pre-defined genomic location of the cell, comprising:
III. Providing the cell isolated in the preceding step, which cell comprises the nucleic acid sequence I1 integrated at the pre-defined genomic location;
IV. Performing step ii), steps iii)-iv), step v), step vi), and step vii);
(C) Provided that n is larger than the number of additional donor vectors integrated at the pre-defined genomic location of the cell isolated in the preceding step, integrating an additional donor vector into the pre-defined genomic location of the cell, comprising:
III. Providing the cell obtained by performing the preceding step (B), which cell comprises the nucleic acid sequence I3 integrated at the pre-defined genomic location;
IV. Repeating steps (A)II to (A)IX and step (B) until the cell has n additional donor vectors integrated at the pre-defined genomic location.

12. The method of claim 1, wherein:
the cell of step i) comprises n pre-defined genomic locations, each of which comprises a nucleic acid sequence I, I1₁ to I1ₙ, respectively, wherein the I1₁ to I1ₙ are different from each other;
step ii) comprises providing 1 to n donor vectors, each of the 1 to n donor vectors comprising a nucleic acid sequence I2₁ to I2ₙ, respectively, which is capable of recombining with the corresponding I1₁ to I1ₙ nucleic acid sequence of said donor vector in the presence of said first DNA enzyme, and each of the 1 to n donor vectors comprising a first selection marker, SM₁ to SMₙ, wherein the SM₁ to SMₙ are different from each other;
step iv) comprises introducing the 1 to n donor vectors into the cell;
step v) comprises selecting a cell having each of the 1 to n donor vectors integrated at its corresponding pre-defined genomic location by detecting each of the different first selection markers, SM₁ to SMₙ, in the cell;
wherein n is an integer ≥2.

13. The method of claim 8, wherein the donor vector of step ii) further comprises:
f. a nucleic acid sequence I3 comprising a recognition site for a first DNA enzyme; and;
g. a promotor nucleic acid sequence P2, and
wherein the excised sequence comprises the expression cassette containing a gene encoding protein of interest.

14. The method of claim 1, wherein said first DNA enzyme is a recombinase.

15. The method of claim 14, wherein:
(a) I1 comprises two recombinase recognition site variants I1a and I1b; and
(b) I2 comprises two recombinase recognition site variants I2a and I2b; and
(c) I1a is capable of recombination with I2a and I1b is capable of recombination with I2b in the presence of said first DNA enzyme.

16. The method of claim 15, wherein I1a is identical to I2a and I1b is identical to I2b.

17. The method of claim 16, wherein I1a, I1b, I2a and I2b are selected from loxP, rox or FRT or variants thereof, respectively, and wherein the first DNA enzyme is selected from the group consisting of a Cre recombinase, a Dre recombinase and a FLP recombinase, respectively.

18. The method of claim 14, wherein:
(a) I1 comprises a single recombinase recognition site; and
(b) I2 comprises a single recombinase recognition site; and
(c) I1 and I2 are capable of recombination in the presence of said first DNA enzyme.

19. The method of claim 18, wherein the recombinase recognition site comprised by I1 differs in sequence from the recombinase recognition site comprised by I2.

20. The method of claim 19, wherein said recombinase recognition sites are selected from attB, attP or a variant thereof.

21. The method of claim 18, wherein said recombinase is a PhiC31 or Bxb1 recombinase or a mutant thereof.

22. The method of claim 1, wherein said first DNA enzyme is a gene editing nuclease.

23. The method of claim 22, wherein:
(a) I1 comprises a cut site for said gene editing nuclease and two sequence regions LHA1 and RHA1; and
(b) I2 comprises two sequence regions LHA2 and RHA2 homologous to LHA1 and LHA2; and (c) I1 and I2 are capable of recombination in the presence of said first DNA enzyme.

24. The method of claim 22, wherein said gene editing enzyme is selected from the group consisting of (i) zink finger nucleases (ZFNs); (ii) homing endo nucleases, such as meganucleases; (iii) TALENS and (iv) DNA or RNA guided nucleases, such as CRISPR/Cas 9.

25. The method of claim 1, wherein the nucleic acid sequences E1 and E2 are identical recombinase recognition sites, such as loxP, rox or FRT or variants thereof, respectively, provided that E1 and E2 are different from I1 and I2.

26. The method of claim 24, wherein said second DNA enzyme is selected from the group consisting of a Cre recombinase, a Dre recombinase and a FLP recombinase, provided that the first DNA enzyme is not a Cre recombinase, a Dre recombinase or a FLP recombinase.

27. The method of claim 1, wherein the promotor nucleic acid sequence P1 and/or P2 when integrated at said pre-defined genomic location is functionally fused to the 5′-part of a split intron.

28. The method of claim 1, wherein said excised nucleic acid sequence comprise;
(a) Said nucleic acid sequence encoding a first selection marker;
(b) Said promotor nucleic acid sequence P1 or P2; and/or
(c) Said expression cassette encoding a second selection marker.

29. The method of claim 1, wherein said first selection marker is selected from the groups of (i) fluorescent proteins and (ii) heterologous cell surface markers.

30. The method of claim 1, wherein the first DNA enzyme is provided in the form of a plasmid, mRNA or a purified protein, optionally wherein said first DNA enzyme is encoded by and expressed from said donor vector.

31. The method of claim 1, wherein the second DNA enzyme is provided in the form of a plasmid, mRNA or a purified protein.

32. The method of claim 1, wherein said donor vector of step ii) further comprises an expression cassette encoding a second DNA enzyme, the expression of said second DNA enzyme being activated when said donor vector has been integrated into a pre-defined genomic location of a cell of step i).

33. The method of claim 1, wherein the first DNA enzyme is expressed from an expression cassette encoding said first DNA enzyme present in the pre-defined genomic location of a cell of step i).

34. The method of claim 1, wherein said eukaryotic cell is selected from the group consisting of a yeast cell, a filamentous fungus cell, a plant cell, an insect cell or a mammalian cell.

35. An isolated eukaryotic cell obtainable by the method of claim 1.

36. A method for producing a recombinant protein, said method comprising:
i) obtaining an isolated eukaryotic cell comprising one or more nucleic acid sequences of interest integrated at a pre-defined genomic location by performing the method of claim 1, wherein at least one nucleic acid sequence of interest comprises at least one expression cassette comprising a gene encoding a protein of interest;
ii) in said cell of step i), producing a protein encoded by the gene of interest; and
iii) isolating the protein of step ii).
